(19) United States
(12) Patent Application Publication
(10) Pub. No.: US 2026/0022399 A1
(43) Pub. Date: Jan. 22, 2026

(54) AAV-BASED TREATMENT FOR ALAGILLE SYNDROME

(71) Applicants: Baylor College of Medicine, Houston, TX (US); University of Massachusetts, Westborough, MA (US)

(72) Inventors: Hamed Jafar-Nejad, Houston, TX (US); Duncan Fox, Houston, TX (US); Guangping Gao, Worcester, MA (US); Jun Xie, Worchester, MA (US)

(73) Assignees: Baylor College of Medicine, Houston, TX (US); University of Massachusetts, Westborough, MA (US)

(21) Appl. No.: 19/147,427

(22) PCT Filed: Jan. 15, 2024
(86) PCT No.: PCT/US2024/011551

§ 371 Date: Jul. 11, 2025

Related U.S. Application Data
(60) Provisional application No. 63/480,075, filed on Jan. 16, 2023.

(51) Int. Cl. C12N 15/86; A61P 1/16; C12N 15/113

(52) U.S. Cl. CPC C12N 15/86; A61P 1/16; C12N 15/113; C12N 2310/531; C12N 2750/14143

(57) ABSTRACT

Embodiments of the disclosure encompass methods and compositions related to treatment for individuals that have bile duct paucity, such as individuals with Alagille Syndrome. The methods and compositions relate to use of inhibitory agents that target SOX4 expression to reduce it or that target SOX4 or to reduce its activity. Specific embodiments include AAV8 vectors that encode an shRNA that targets SOX4.

### BACKGROUND

This application claims priority to U.S. Provisional Patent Application Ser. No. 63/480,075, filed on Jan. 16, 2023, which is incorporated by reference herein in its entirety.

### I. Technical Field

This disclosure relates at least to the fields of cell biology, molecular biology, physiology, hepatology, the biliary system, and medicine.

### II. BACKGROUND

Alagille syndrome (ALGS) is a multi-system disorder affecting the liver, cardiovascular system, kidneys, eye, skeleton and other organs (1, 2). The hallmark of the disease is bile duct paucity, or a significant decrease in the number of bile ducts (BD) per portal vein (PV) on average (2). BD paucity results in intrahepatic cholestasis, or the accumulation of bile in the liver, in many ALGS patients and often results in liver failure. ALGS is caused by autosomal dominant mutations in the Notch signaling pathway components, with JAG1 accounting for 95% of cases and NOTCH2 for 2-3% (3-8).

With an estimated frequency of 1:30,000, ALGS is the most common genetic disease resulting in intrahepatic cholestasis (9). Standard of clinical care for BD paucity is directed towards reducing intrahepatic bile accumulation by altering the flow through surgery or modifying the enterohepatic bile acid transport through inhibitors and binding resins (10, 11). However, similar to other diseases associated with severe BD paucity, the only cure for the liver disease in ALGS is liver transplantation (11). The shortage of suitable liver donors and the rather long time that patients usually spend on the liver transplant wait list highlight the need for identifying strategies to bypass the requirement for liver transplantation. This is especially the case in ALGS due to 1) the severity of coexisting cardiovascular and renal anomalies, which exclude some ALGS patients from liver transplantation, 2) the long-term adverse effects of immunosuppressive therapy after pediatric liver transplantation, and 3) relatively high rate of complications after liver transplantation in ALGS patients (12, 13).

A meta-analysis of 20 papers on ALGS epidemiology and natural history found that 15%-47% of ALGS patients underwent liver transplant (14). However, these studies were from mixed cohorts of patients, some with and some without significant liver disease. A recent study followed 293 ALGS patients who showed features of cholestasis by the age of 5 and had their native liver at the time of enrollment (15). Prospective analysis of these patients showed that only 24% of them survived to age 18.5 without a liver transplant. This work, which is the largest multi-center natural history study of the ALGS liver disease to date, also reported that ALGS patients exhibit a second wave of liver disease due to fibrosis and portal hypertension in later childhood (11, 15). A more recent multicenter retrospective study analyzed the natural history of the disease for 1,433 patients from 29 countries who were clinically and/or genetically confirmed to have ALGS (16). In the 1,184 patients who had a history of neonatal cholestasis, a native liver survival rate of 40.3% at 18 years of age was reported (16). Moreover, 68.9% of patients developed clinically evident portal hypertension by the age of 18 (16). Together, these studies established that the burden of the liver disease and the need for transplantation in ALGS patients is even higher than previously appreciated.

The present disclosure satisfies a long-felt need in the art to provide therapeutic relief for patients with bile duct paucity, such as ALGS.

### SUMMARY

Embodiments of the disclosure encompass compositions and methods related to treatment for an individual with an insufficient number of bile ducts associated with the liver of the individual. Embodiments of the disclosure encompass compositions and methods related to treatment for the accumulation of bile in the liver of an individual. Embodiments of the disclosure encompass compositions and methods related to treatment for liver failure of an individual. Embodiments of the disclosure encompass compositions and methods related to treatment for an individual at risk for liver failure. Embodiments of the disclosure encompass compositions and methods related to treatment to avoid the need for liver transplant for an individual or reduce the risk for need for liver transplant for an individual. Embodiments of the disclosure encompass compositions and methods related to prevention of liver failure for an individual with autosomal dominant mutations in the JAG1 gene or the NOTCH2 gene or any other gene(s) associated with ALGS. Embodiments of the disclosure encompass compositions and methods related to reducing the accumulation of bile in an individual with an insufficient number of bile ducts associated with the liver in the individual. Embodiments of the disclosure encompass compositions and methods related to reducing the accumulation of bile in an individual with autosomal dominant mutations in the JAG1 gene or the NOTCH2 gene or any other gene(s) associated with ALGS. Embodiments of the disclosure encompass compositions and methods related to preventing or delaying the onset of deleterious accumulation of bile in the liver and/or increasing the number of bile ducts of an individual with an insufficient number of bile ducts associated with the liver of the individual. Embodiments of the disclosure encompass compositions and methods related to delaying the onset of deleterious accumulation of bile in the liver of an individual with autosomal dominant mutations in the JAG1 gene or the NOTCH2 gene or any other gene(s) associated with ALGS.

Any embodiments encompassed herein may include one or more of the following steps in any order: administering to an individual one or more agents that inhibit SOX4 gene expression and/or SOX4 protein activity; administering to an individual one or more nucleic acids that inhibit fully or partially expression of SOX4 gene; administering to an individual one or more proteins that inhibit fully or partially expression of SOX4 gene and/or activity of SOX4 protein; administering to an individual one or more antibodies that inhibit fully or partially SOX4 protein activity; administering to an individual one or more small molecules that inhibit fully or partially SOX4 protein activity; preparing one or more agents that inhibit SOX4 gene expression and/or SOX4 protein activity; preparing one or more nucleic acids that inhibit fully or partially expression of SOX4 gene; preparing one or more proteins that inhibit fully or partially expression of SOX4 gene and/or activity of SOX4 protein; preparing one or more antibodies that inhibit fully or partially SOX4 protein activity; preparing one or more small molecules that inhibit fully or partially SOX4 protein activity; assaying a sample from an individual suspected of having bile duct paucity; assaying a sample from an individual known to have bile duct paucity; assaying a sample from an individual with a biological parent that has bile duct paucity; assaying a sample from an individual for a mutation in the JAG1 gene or NOTCH2 gene; assaying serum bile acid profiles from an individual suspected of having or known to have bile accumulation in the liver; assaying serum bile acid profiles from an individual suspected of having or known to have a mutation in the JAG1 gene or NOTCH2 gene; and measuring the level of one or more of the following from a sample from an individual suspected of having or known to have bile accumulation in the liver, or from an individual suspected of having or known to have a mutation in the JAG1 gene or NOTCH2 gene: bilirubin, alkaline phosphatase, 5′-nucleotidase, gamma-glutamyl transpeptidase, cholic acid, chenodeoxycholic acid, taurocholic acid, glycocholic acid, glycochenodeoxycholic acid, taurochenoxycholic acid, deoxycholic acid, glycodeoxycholic acid, taurodeoxycholic acid, deoxy-2,2,4,4-d4 acid, Cholic-2,2,4,4-d4 acid, glycocholic-2,2,4,5-d4 acid, chenodeoxycholic-2,2,4,4-d4 acid, glycochenodeoxycholic-2,2,4,4-d4 acid, Taurocholic 2,2,3,4,4-d5 acid, and taurochenodeoxycholic-2,2,3,4,4-d5 acid. Measurement of such elements may occur by radioimmunoassay, liquid chromatography-mass spectrometry, liquid chromatography with tandem mass spectrometry, etc. Any sample referred to herein may be of any kind, including liver biopsy and/or serum, for example.

Embodiments of the disclosure concern a method of treating bile duct paucity in an individual, comprising administering to the individual an effective amount of one or more agents that reduces expression of SRY-Box Transcription Factor 4 (SOX4) gene and/or activity of SOX4 protein. In specific embodiments, the individual has neonatal cholestasis and/or Alagille syndrome (ALGS). The agent may partially or fully reduce expression of SOX4. The agent may be a nucleic acid, protein, small molecule, or combination thereof. In specific embodiments, the agent is a nucleic acid that targets expression of SOX4. The agent may be an shRNA that targets expression of SOX4. The agent may be a SOX4 antibody or functional fragment thereof, including an antibody that is polyclonal or monoclonal. The individual may be of any kind, including in utero or is a neonate, infant, child, or adult. The individual may have cholestasis, fibrosis, necrosis, and/or ductular reactions. The individual may be in need of a liver transplant or is at risk for needing a liver transplant. The individual may have liver failure. The individual may or may not have an autosomal dominant mutation in the JAG1 gene, the NOTCH2 gene, or another gene(s) associated with ALGS. The individual may have had surgery for bile duct paucity or will have surgery for bile duct paucity. Enterohepatic bile acid transport in the individual may have been or will be modified through one or more inhibitors and/or one or more binding resins. The individual may have affected the cardiovascular system, kidneys, eye, and/or skeleton of the individual.

In specific embodiments, the one or more agents is a nucleic acid in a vector, such as a viral or non-viral vector. In certain embodiments, the viral vector is an adeno-associated viral vector, an adenoviral vector, a lentiviral vector, or a retroviral vector. The adeno-associated viral vector may be AAV8, AAV9, or AAV3b.

It is contemplated that any embodiment discussed in this specification can be implemented with respect to any method or composition of the invention, and vice versa. Furthermore, compositions of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure. The subject matter of the disclosure may be better understood by reference to one or more of these drawings in combination with the detailed description of specific embodiments presented herein.

FIGS. 1A-1D. Conditional removal of one copy of Sox4 in the livers of Jag1^{+/-} mice improves their BD development. (1A) P30 liver sections stained with DAPI and wide spectrum cytokeratin (wsCK). Asterisks mark PVs, arrowheads mark patent BDs. Scale bar is 50 m. Sox4^{+/f} represents Sox4^{+/flox}. Mutant alleles are marked in red in this and other figure. (1B) Quantification of number of BDs per PV. (1C) Sirius Red staining of the P30 livers with indicated genotypes. Scale bar is 100 μm. (1D) Quantification of number of BDs per PV. (1D) Quantification of the percent Sirius Red⁺ area of each liver. In 1B and 1D, each circle is one animal. Mean±standard deviation is shown. NS: not significant, ****P<0.0001.

FIGS. 2A-2B. Conditional removal of one copy of Sox4 in the livers of Jag1^{+/-} mice improves their biliary tree formation. (2A) Biliary tree ink injection images from left liver lobes of P30 and P60 animals with indicated genotypes. Given the variability of the Jag1^{+/-} phenotype, for Jag1^{+/-}; Sox4^{+/fl} and Jag1^{+/-}; Sox4^{+/Δ} genotypes at each age, two images at the best and worst end of the phenotypic spectrum are shown. (2B) Quantification of the percent area covered by the ink-filled biliary tree in each left liver lobe. Biliary tree density was calculated in a frame of the same size from the hilar region of each left lobe. Each circle is one animal. Mean±standard deviation is shown. NS: not significant, ***P<0.001, ****P<0.0001.

FIGS. 3A-3F. Conditional removal of one copy of Sox4 in the livers of Jag1^{+/-}; Sox9^{+/Δ} mice improves their BD development and biliary tree formation. (3A) P30 liver sections of indicated genotypes stained with DAPI and wide spectrum cytokeratin (wsCK). Asterisks mark PVs, arrowheads mark patent BDs. Scale bar is 50 μm. (3B) Quantification of number of BDs per PV. (3C) Sirius Red staining of the P30 livers with indicated genotypes. Note that many areas in Jag1^{+/-}; Sox9^{+/Δ}; Sox4^{+/Δ} livers show no fibrosis at all (not shown). Scale bar is 100 μm. (3D) Quantification of the percent Sirius Red⁺ area of each liver. (3E) Biliary tree ink injection images from left liver lobes of P30 animals with indicated genotypes. (3F) Quantification of the percent area covered by the ink-filled biliary tree in each left liver lobe. Biliary tree density was calculated in a frame of the same size from the hilar region of each left lobe. In 3B, 3D and 3F, each circle is one animal. Mean±standard deviation is shown. NS: not significant,. NS: not significant, *P<0.05, **P<0.01, ***P<0.001, ****P<0.0001.

FIGS. 4A-4B. Generation of AAV8 vectors capable of in vivo Sox4 knockdown. (4A) Schematic of the vectors used in our studies. (4B) qRT-PCR shows that a single IP injection of AAV8-CB6-amiR-Sox4 (ubiquitous promoter) and AAV8-TBG-amiR-Sox4 (only in TBG+ cells) both lead to significant Sox4 KD in mouse livers. Note that Jag1^{+/-} livers express higher Sox4 mRNA levels compared to WT. Each circle represents an animal; Horizontal lines show mean±standard deviation. One-way ANOVA with Tukey multiple comparisons test was used for analyzing the data for each genotype. NS, not significant, *P<0.05.

FIGS. 5A-5H. AAV8-mediated knockdown of Sox4 in the livers of Jag1^{+/-} mice improves their liver phenotypes. (5A) P30 liver sections of indicated genotypes stained with DAPI and wide spectrum cytokeratin (wsCK). Asterisks mark PVs, arrowheads mark patent BDs. Scale bar is 50 μm. (5B) Sirius Red staining of the P30 livers with indicated genotypes. Scale bar is 100 μm. (5C) H&E staining of the P30 livers with indicated genotypes. Note the areas of necrosis and hypercellularity in the Jag1^{+/-} liver from the control animal injected with AAV8-CB6-eGFP. Scale bar is 100 μm. (5D) Biliary tree ink injection images from left liver lobes of P30 animals with indicated genotypes. (5E) Quantification of number of BDs per PV. (5F) Quantification of the percent Sirius Red⁺ area of each liver. (5G) Quantification of the percent area covered by necrosis in each left liver lobe based on H&E staining images. (5H) Quantification of the percent area covered by the ink-filled biliary tree in each left liver lobe. Biliary tree density was calculated in a frame of the same size from the hilar region of each left lobe. In 5E-5H, each circle is one animal. Mean±standard deviation is shown. NS: not significant, *P<0.05, **P<0.01, ***P<0.001, ****P<0.0001.

FIGS. 6A-6F. AAV8-mediated knockdown of Sox4 in the livers of Jag1^{+/-}; Sox9^{+/Δ} mice improves their liver histology. (6A) P30 liver sections of indicated genotypes stained with DAPI and wide spectrum cytokeratin (wsCK). Asterisks mark PVs, arrowheads mark patent BDs. Scale bar is 50 μm. (6B) Sirius Red staining of the P30 livers with indicated genotypes. Scale bar is 100 μm. (6C) H&E staining of the P30 livers with indicated genotypes. Note the areas of necrosis and hypercellularity in the Jag1^{+/-}; Sox9^{+/Δ} liver from the control animal injected with AAV8-CB6-eGFP. Scale bar is 100 μm. (6D) Quantification of number of BDs per PV. (6E) Quantification of the percent Sirius Red⁺ area of each liver. (6F) Quantification of the percent area covered by necrosis in each left liver lobe based on H&E staining images. In 6D-6F, each circle is one animal. Mean±standard deviation is shown. Two-way ANOVA with Tukey multiple comparisons test was used. NS: not significant, *P<0.05, **P<0.01, ***P<0.001, ****P<0.0001.

FIGS. 7A-7E. AAV8-mediated knockdown of Sox4 in the livers of Jag1^{+/-}; Sox9^{+/Δ} mice improves their biliary tree structure and rescues their liver damage and lethality. (7A) Biliary tree ink injection images from left liver lobes of P30 animals with indicated genotypes and AAV injections. (7B) Quantification of the percent area covered by the ink-filled biliary tree in each left liver lobe. Biliary tree density was calculated in a frame of the same size from the hilar region of each left lobe. (7C,7D) Serum levels of AST (7C) and ALP (7D) in the indicated genotypes and AAV injections. (7E) Survival of animals with the indicated genotypes and AAV injections. AAV8-CB6-eGFP was used as control (amiR-Sox4−). In 7B-7D, each circle is one animal. Mean±standard deviation is shown. Two-way ANOVA with Tukey multiple comparisons test was used. In 7E, n=number of animals. Kaplan Meier was used. NS: not significant, *P<0.05, **P<0.01, ***P<0.001, ****P<0.0001.

FIG. 8. AAV8-amiR-Sox4 vectors do not lead to liver abnormalities in control animals. Biliary tree ink injection images from left liver lobes of animals ˜P150 (five months of age) with indicated genotypes and AAV injections. At least 3 animals per genotype/injection condition were analyzed.

FIGS. 9A-9C. AAV8-amiR-Sox4 vectors do not lead to liver abnormalities in control animals. (9A) P30 liver sections of indicated genotypes stained with DAPI and wide spectrum cytokeratin (wsCK). Asterisks mark PVs, arrowheads mark patent BDs. Scale bar is 50 μm. (9B) Sirius Red staining of the P30 livers with indicated genotypes. Scale bar is 100 m. (9C) Biliary tree ink injection images from left liver lobes of P30 animals with indicated genotypes and AAV injections. For quantifications, please see FIGS. 5E, SF, 5H, 6D, 6E, and 7B.

FIGS. 10A-10B. AAV8-mediated knockdown of Sox4 by a second amiR-Sox4 or by retro-orbital injection can also rescue the Jag1+/− liver phenotypes. (10A, 10B) Biliary tree ink injection images from left liver lobes of P30 animals with indicated genotypes and AAV injections. Each image is representative of at least 3 animals.

### DETAILED DESCRIPTION

### I. Examples of Definitions

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

The term “effective,” as that term is used in the specification and/or claims, means adequate to accomplish a desired, expected, or intended result.

The terms “individual,” “subject,” and “patient” are used interchangeably and can refer to a human or non-human.

The terms “inhibiting” or “reducing” or “preventing” or “avoiding” or any variation of these terms, when used in the claims and/or the specification, includes any measurable decrease or complete inhibition to achieve a desired result.

As used herein, a “protein” or “polypeptide” refers to a molecule comprising at least five amino acid residues. As used herein, the term “wild-type” refers to the endogenous version of a molecule that occurs naturally in an organism. In some embodiments, wild-type versions of a protein or polypeptide are employed, however, in many embodiments of the disclosure, a modified protein or polypeptide is employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some embodiments, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity.

Where a protein is specifically mentioned herein, it is in general a reference to a native (wild-type) or recombinant (modified) protein or, optionally, a protein in which any signal sequence has been removed. The protein may be isolated directly from the organism of which it is native, produced by recombinant DNA/exogenous expression methods, or produced by solid-phase peptide synthesis (SPPS) or other in vitro methods. RNA encoding the protein, from a linear or circular nature, can be produced synthetically or by in vitro methods. In particular embodiments, there are isolated nucleic acid segments and recombinant vectors incorporating nucleic acid sequences that encode a polypeptide. The term “recombinant” may be used in conjunction with a polypeptide or the name of a specific polypeptide, and this generally refers to a polypeptide produced from a nucleic acid molecule that has been manipulated in vitro or that is a replication product of such a molecule.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

The term “about” as used herein refers to include the usual error range for the respective value readily known. Reference to “about” a value or parameter herein includes (and describes) embodiments that are directed to that value or parameter per se. For example, description referring to “about X” includes description of “X”. In some embodiments, “about” may refer to ±15%, ±10%, 5%, or ±1% as understood by a person of skill in the art.

The term “synthetic” or “synthesized” or any variation of these terms, as used herein refers to molecules that are designed and produced by a person skilled in the art. Methods of producing a synthetic molecule comprise chemical, in vitro, in cellulo, and/or any combination thereof. Methods of producing a synthetic molecule may further comprise selection, purification, concentration, and/or any combination thereof.

The term “helper virus” as used herein refers to viruses that when co-infected into and/or co-expressed in a host cell, supports AAV replication, AAV gene expression, and/or AAV virion production.

The term “helper gene(s),” and “helper factor(s)” are used interchangeably herein and refer to genes, RNAs, and/or proteins derived from helper viruses and/or cells that support AAV replication, AAV gene expression, and/or AAV virion production.

The term “recombinant” may be used in conjunction with a polypeptide or the name of a specific polypeptide, and this generally refers to a polypeptide produced from a nucleic acid molecule that has been manipulated in vitro or that is a replication product of such a molecule.

It is specifically contemplated that any limitation discussed with respect to one embodiment of the invention may apply to any other embodiment of the invention. Furthermore, any composition of the invention may be used in any method of the disclosure, and any method of the invention may be used to produce or to utilize any composition of the disclosure. Any embodiment discussed with respect to one aspect of the disclosure applies to other aspects of the disclosure as well and vice versa. For example, any step in a method described herein can apply to any other method. Moreover, any method described herein may have an exclusion of any step or combination of steps. Aspects of an embodiment set forth in the Examples are also embodiments that may be implemented in the context of embodiments discussed elsewhere in a different Example or elsewhere in the application, such as in the Summary, Detailed Description, Claims, and Brief Description of the Drawings.

### II. General Embodiments

Alagille syndrome leads to a severe reduction in the number of bile ducts in the liver (bile duct paucity) and is the most common genetic cause of intrahepatic cholestasis. There are no approved treatments to address the underlying cause of Alagille syndrome, and a majority of patients will need a liver transplant by age 18. To address the large gap in treatment, the inventors have developed a method of treating bile duct paucity using an AAV8 vector containing an shRNA targeting SOX4. Postnatal reduction of SOX4 in the liver by this AAV-based treatment is able to improve the bile duct paucity and subsequent liver damage in two mouse models of Alagille syndrome with moderate to severe bile duct abnormalities. Therefore, this disclosure presents studies supporting a novel treatment for Alagille syndrome, which likely averts the need for a liver transplant in this patient population. In particular embodiments, there are no approved therapies for any disease that target SOX4, providing additional utilities for the subject matter of the present disclosure.

As shown herein, in order to address the significant gap in treatment of Alagille syndrome (ALGS) liver disease, a mouse model of ALGS was utilized to study its progression and identify candidate therapeutic targets. By crossing a previously established Jag1 heterozygous deletion (Jag1^{+/-}; (17)) onto a C57BL6 genetic background, a mouse line with BD paucity beginning before birth and persisting into adulthood was generated (18). Jag1^{+/-} animals show phenotypes consistent with the liver disease in ALGS, including the clinically relevant phenotypes cholestasis, fibrosis, necrosis, and ductular reactions (18, 19). In order to determine mechanism(s) underlying the phenotypic variability in ALGS patients, the Jag1^{+/-} model was used to identify genetic modifiers of the Jag1 haploinsufficient phenotypes, and it was reported that the Jag1^{+/-} liver phenotypes are highly sensitive to the level of the transcription factor Sox9 (19). Genetic experiments showed that removal of one or both copies Sox9 from the liver using the Albumin-Cre transgene (20) results in a stepwise worsening of Jag1^{+/-} liver phenotypes and allowed establishment of additional ALGS models: (Jag1^{+/-}; Sox9^{+/flox}; Albumin-Cre [hereafter called Jag1^{+/-}; Sox9^{+/Δ}] and Jag1^{+/-}; Sox9^{flox/flox}; Albumin-Cre [hereafter called Jag1^{+/-}; Sox9^{Δ/Δ}]) (19). Notably, the BD paucity in these two models is more severe than the original Jag1^{+/-} model. Moreover, while the Jag1^{+/-} liver phenotypes somewhat improve as the animals age, Jag1^{+/-}; Sox9^{+/Δ} animals do not exhibit any improvement in liver phenotypes upon aging, (19). Overexpression of Sox9 with Albumin-Cre leads to a full and sustained rescue of the Jag1-haploinsufficient liver phenotypes without any side effects (19). Finally, analysis of SOX9 expression in human livers showed that ALGS patients with mild liver disease express higher levels of SOX9 compared to ALGS patients with severe liver disease (19), providing additional support for the role of SOX9 in determining the severity of ALGS liver disease.

In specific embodiments, the data indicate that increasing the level of SOX9 has therapeutic benefits in ALGS patients. However, given the potential side effects of overexpressing a transcription factor like Sox9 as a treatment strategy, instead the Jag1^{+/-} and Jag1^{+/-}; Sox9^{+/Δ} mouse models were employed to identify additional therapeutic candidates for ALGS liver phenotypes. Herein it is demonstrated that removing one copy of Sox4 in the liver with Albumin-Cre results in improvement of phenotypes in both of these ALGS models. Because Albumin-Cre induces recombination during the embryonic period (21), the above-mentioned genetic studies were not sufficient to establish the utility of reducing Sox4 as a therapeutic strategy in ALGS patients. Therefore, two adeno-associated virus 8 (AAV8) vectors were designed and produced expressing an artificial miRNA that carries a short hairpin RNA (shRNA) that targets Sox4 and is able to reduce Sox4 levels in vivo, one ubiquitously and the other in a liver-specific fashion. A third adeno-associated virus 8 (AAV8) vector was designed and produced expressing an artificial miRNA that carries an independent short hairpin RNA (shRNA) that targets Sox4 and is able to reduce Sox4 levels in vivo ubiquitously. As shown herein, a single postnatal injection of any of these AAV8 constructs is able to dramatically improve ALGS liver phenotypes in mouse models, thereby providing strong preclinical evidence that reducing SOX4 levels is a viable option for improving the liver phenotypes and averting the need for liver transplantation in ALGS patients.

Embodiments of the disclosure include methods of treating individuals of any age with bile duct paucity by administering to the individual one or more agents that reduce expression of SOX4 gene and/or that reduce activity of SOX4 protein. Embodiments of the disclosure include methods of treating individuals of any age with cholestasis (including neonatal cholestasis) by administering to the individual one or more agents that reduce expression of SOX4 gene and/or that reduce activity of SOX4 protein. In specific embodiments, the disclosure provides methods of treating individuals of any age with ALGS with one or more agents that reduce expression of SOX4 gene and/or that reduce activity of SOX4 protein. The individual may be in need of a liver transplant or is at risk for needing a liver transplant and/or has liver failure, and/or the individual may be affected in the cardiovascular system, kidneys, eye, and/or skeleton. In specific embodiments, the individual is a neonate, infant, child, or adult. The individual may or may not be an infant that is, or is less than, 4, 3, 2, or 1 week old. The individual may be an infant that is, or is less than, 12, 11, 10, 9, 8, 7, 6, 5, 4, 3, 2, or 1 month old. The individual may be a child or adolescent that is, or is less than, 17, 16, 15, 14, 13, 12, 11, 10, 9, 8, 7, 6, 5, 4, 3, 2, or 1 year old.

Reduction in expression or activity in any embodiment may or may not be complete. The agent may be of any kind, such as a nucleic acid, protein, small molecule, carbohydrate, or combination thereof. In specific embodiments, the agent is a nucleic acid (such as a shRNA) that targets expression of SOX4, although the agent may be a SOX4 antibody. In cases wherein the agent is a nucleic acid, the nucleic acid may be administered in a vector of any kind, including a viral vector or a non-viral vector. In specific embodiments, the vector is an adeno-associated virus 8 (AAV8) vector, an AAV3b vector, or an AAV9 vector, each that has proficient capabilities to transduce hepatocytes.

### III. Agents that Reduce Expression of SOX4 Gene and/or Activity of SOX4 Protein

In specific embodiments, an individual in need thereof is administered an effective amount of one or more agents that reduce expression of SOX4 gene and/or activity of SOX4 protein. The reduction of expression and/or activity may be partial or may be complete, although the data indicate that even a partial reduction is sufficient to significantly improve the phenotypes. In specific embodiments, when more than one agent is administered, they may or may not be administered at the same time. In specific embodiments, when more than one agent is administered at the same time, they may or may not be in the same composition. In specific embodiments, when more than one agent is administered at the same time or at different times, their route of administration may not be the same.

In particular embodiments, the one or more agents that reduce expression of SOX4 gene and/or activity of SOX4 protein in an individual include one or more nucleic acids (DNA and/or RNA), one or more proteins, one or more small molecules, one or more carbohydrates, or a combination thereof.

### A. Nucleic Acids, Generally

In certain embodiments, nucleic acid sequences for use in the invention can exist in a variety of instances such as: isolated segments and recombinant vectors of incorporated sequences or recombinant polynucleotides encoding one or both chains of an antibody, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing described herein. The nucleic acids can be single-stranded or double-stranded and can comprise RNA and/or DNA nucleotides and artificial variants thereof (e.g., peptide nucleic acids).

The term “polynucleotide” refers to a nucleic acid molecule that either is recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar protein.

In certain embodiments, there are polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence provided herein using the methods described herein (e.g., BLAST analysis using standard parameters). In certain aspects, the isolated polynucleotide will comprise a nucleotide sequence encoding a polypeptide that has at least 90%, preferably 95% and above, identity to an amino acid sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, 5, 10, 15, 20, 25, 30, 35, 40, 45, 50, 75, 100, 125, 175, 200, 250, 300, 350, 400, 450, 500, 750, 1000, 1500, 3000, 5000 or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

### IV. RNA Interference

In particular embodiments, an individual encompassed herein is provided agents for RNA interference to reduce expression of SOX4. RNA interference (RNA interference, RNAi) refers to the phenomenon that double-stranded RNA molecules (dsRNA) enter human cells and specifically degrade the homologous mRNA, thereby specifically and efficiently inhibiting the expression of corresponding genes. RNA interference was first discovered in plants and lower organisms. With the deepening of research, it has also been discovered in higher eukaryotes recently, and it has proved to be an important evolutionary conservation phenomenon. When the homologous dsRNA with the coding region of endogenous mRNA is introduced into the cell, the mRNA will be degraded and the gene expression will be silenced, which is a special kind of post transcriptional gene silence (PTGS). In treatment, RNAi works by delivering small RNA duplexes, including microRNA (miRNA) mimics, small interfering RNA (siRNA), short hairpin RNA (shRNA) and Dicer substrate RNA (dsiRNA). It has been proved that siRNA is cleaved by dsRNA-specific endonuclease (Dicer enzyme). RNA-induced silencing complex (RISC) is composed of siRNA and a multi-enzyme complex. It is located in a specific part of mRNA and exerts the activities of endonuclease and exonuclease to act on mRNA. In addition to gene silencing (degrading mRNA) at the transcriptional level, siRNA has also been shown to reduce protein expression by silencing the promoter by DNA methylation.

Because RNAi has a high degree of sequence specificity and effective interference, it can specifically silence specific genes, thereby obtaining gene function loss or gene expression reduction, so this technology has been widely used to explore gene function, cancer and other diseases.

In particular embodiments, any type of RNA interference is utilized to target the SOX4 gene such as is represented by SEQ ID NO:1 (see below).

A. siRNA

Small interfering RNA (siRNA), sometimes called short interfering RNA or silencing RNA, is a type of double-stranded RNA molecule with a length of 20-25 base pairs, similar to miRNA, and operates within the RNA interference (RNAi) pathway. It interferes with post-transcriptionally degraded mRNA of specific genes expressing complementary nucleotide sequences, thereby preventing translation. siRNA is a double-stranded RNA (double strand RNA, dsRNA) that is cleaved by RNase III (such as Dicer) into double-stranded RNA with a size of 21-25 bp in the cell. In one aspect of the present invention, there is provided an siRNA that inhibits SOX4.

B. shRNA

Short hairpin RNA (shRNA) includes two short inverted repeats. The shRNA cloned into the shRNA expression vector includes two short inverted repeats, separated by a loop sequence in the middle, forming a hairpin structure. Generally, shRNA expression is controlled by RNA polymerase (Pol) III promoter or modified pol II promoter. Then connect the transcription terminator. After shRNA is transcribed, two short inverted repeats connected by a stem loop form a characteristic hairpin structure together in pairs. In some embodiments, the transcription terminator is 5-6 Ts.

The stem loop in the shRNA insert should be close to the center of the oligonucleotide. Stem loops of different sizes and nucleotide sequences have been successfully used.

shRNA is usually introduced into cells using vectors and can be passed to progeny cells so that gene silencing can be inherited. The hairpin structure of shRNA can be cleaved into siRNA by cellular machinery, and then degrade mRNA according to the aforementioned mechanism. Its biggest advantage is that it has a high degree of effectiveness and specificity, as well as rapid defense and treatment effects. Its role has shown immeasurable value in the field of gene function research and the treatment of various diseases, especially the treatment of viral diseases, such as anti-HIV, hepatitis C virus (HCV), and type B Studies on viruses such as hepatitis virus (HBV) and poliovirus (Poliovirus) have found that RNAi has an excellent effect on inhibiting the replication of these viruses.

In one aspect of the present disclosure, there is provided a shRNA that inhibits SOX4.

In some embodiments, there is a drug that comprises a vector and a nucleic acid sequence encoding a single shRNA. In some embodiments, the shRNA targets a particular sequence of SEQ ID NO: 1. In some embodiments, there is a drug that comprises a vector and a nucleic acid sequence encoding a plurality of shRNAs, wherein the shRNAs individually target different locations of SOX4.

### C. Inhibitory Nucleic Acids

In some aspects, the disclosure relates to inhibitory nucleic acids that inhibit the gene expression of SOX4. Examples of inhibitory nucleic acids include oligonucleotides but are not limited to siRNA (small interfering RNA), short hairpin RNA (shRNA), double-stranded RNA, an antisense oligonucleotide, a ribozyme, and an oligonucleotide encoding any thereof. An inhibitory oligonucleotide may inhibit the transcription of a gene or prevent the translation of a gene transcript in a cell. An inhibitory oligonucleotide acid may be from about 16 to about 1000 nucleotides long, and in certain embodiments from about 18 to about 100 nucleotides long. The oligonucleotide may have at least or may have at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 50, 60, 70, 80, or 90 (or any range derivable therein) nucleotides. The oligonucleotide may be DNA, RNA, or a cDNA that encodes an inhibitory RNA.

As used herein, “isolated” means altered or removed from the natural state through human intervention. For example, an siRNA naturally present in a living animal is not “isolated,” but a synthetic siRNA, or an siRNA partially or completely separated from the coexisting materials of its natural state is “isolated.” An isolated siRNA can exist in substantially purified form, or can exist in a non-native environment such as, for example, a cell into which the siRNA has been delivered.

Inhibitory oligonucleotides are well known in the art. For example, siRNA and double-stranded RNA have been described in U.S. Pat. Nos. 6,506,559 and 6,573,099, as well as in U.S. Patent Publications 2003/0051263, 2003/0055020, 2004/0265839, 2002/0168707, 2003/0159161, and 2004/0064842, all of which are herein incorporated by reference in their entirety.

One representative example of a SOX4 nucleic acid that may be inhibited by an inhibitory nucleic acid is the human SOX4 mRNA represented by GenBank® Accession No. NM_003107 which is as follows:

```
(SEQ ID NO: 1)
      1 gctctaagct gcagcaagag aaactgtgtg tgaggggaag aggcctgttt cgctgtcggg
    61 tctctagttc ttgcacgctc tttaagagtc tgcactggag gaactcctgc cattaccagc
  121 tcccttcttg cagaagggag ggggaaacat acatttattc atgccagtct gttgcatgca
  181 ggctttttgg cttcctacct tgcaacaaaa taattgcacc aactccttag tgccgattcc
  241 gcccacagag agtcctggag ccacagtctt ttttgctttg cattgtagga gagggactaa
  301 gtgctagaga ctatgtcgct ttcctgagct accgagagcg ctcgtgaact ggaatcaact
  361 gcttcaggga aaaagaaaaa aaaaaaaaaa agacttgcct gggaggccgc gagaaacttg
  421 cattggaagc ttcagcaacc agcattcgag aaactcctct ctactttagc acggtctcca
  481 gactcagccg agagacagca aactgcagcg cggtgagaga gcgagagaga gggagagaga
  541 gactctccag cctgggaact ataactcctc tgcgagaggc ggagaactcc ttccccaaat
  601 cttttgggga cttttctctc tttacccacc tccgcccctg cgaggagttg aggggccagt
  661 tcggccgccg cgcgcgtctt cccgttoggc gtgtgcttgg cccggggaac cgggagggcc
  721 cggcgatcgc gcggcggccg ccgcgagggt gtgagcgcgc gtgggcgccc gccgagccga
  781 ggccatggtg cagcaaacca acaatgccga gaacacggaa gcgctgctgg ccggcgagag
  841 ctcggactcg ggcgccggcc tegagetggg aategcctcc tcccccacgc ccggctccac
  901 cgcctccacg ggcggcaagg ccgacgaccc gagctggtgc aagaccccga gtgggcacat
  961 caagcgaccc atgaacgcct tcatggtgtg gtcgcagatc gagcggcgca agatcatgga
1021 gcagtcgccc gacatgcaca acgccgagat ctccaagcgg ctgggcaaac gctggaagct
1081 gctcaaagac agcgacaaga tccctttcat tcgagaggcg gagcggctgc gcctcaagca
1141 catggctgac taccccgact acaagtaccg gcccaggaag aaggtgaagt ccggcaacgc
1201 caactccagc tcctoggccg ccgcctcctc caagccgggg gagaagggag acaaggtcgg
1261 tggcagtggc gggggcggcc atgggggcgg cggcggcggc gggagcagca acgcgggggg
1321 aggaggcggc ggtgcgagtg gcggcggcgc caactccaaa ccggcgcaga aaaagagctg
1381 cggctccaaa gtggcgggcg gcgcgggcgg tggggttagc aaaccgcacg ccaagctcat
1441 cctggcaggc ggcggcggcg gegggaaagc agcggctgcc gccgccgcct ccttcgccgc
1501 cgaacaggcg ggggccgccg ccctgctgcc cctgggegcc gccgccgacc accactcgct
1561 gtacaaggcg cggactccca gegccteggc ctccgcctcc toggcagcct cggcctccgc
1621 agcgctcgcg gccccgggca agcacctggc ggagaagaag gtgaagcgcg tctacctgtt
1681 cggcggcctg ggcacgtcgt cgtcgcccgt gggcggcgtg ggcgcgggag ccgaccccag
1741 cgaccccctg ggcctgtacg aggaggaggg cgcgggctgc togcccgacg cgcccagcct
1801 gagcggccgc agcagegccg cctegtcccc cgccgcegge cgctcgcccg ccgaccaccg
1861 cggctacgcc agcctgegeg ccgcctegcc cgccccgtcc agcgcgccct cgcacgcgtc
1921 ctcctcggcc tegtcccact cctcctette ctcctccteg ggctcctegt cctccgacga
1981 cgagttcgaa gacgacctgc tcgacctgaa ccccagctca aactttgaga gcatgtccct
2041 gggcagcttc agttcgtcgt cggcgctega cegggacctg gattttaact togagcccgg
2101 ctccggctcg cacttcgagt tcccggacta ctgcacgccc gaggtgagcg agatgatctc
2161 gggagactgg ctcgagtcca gcatctccaa cctggttttc acctactgaa gggcgcgcag
2221 gcagggagaa gggccggggg gggtaggaga ggagaaaaaa aaagtgaaaa aaagaaacga
2281 aaaggacaga cgaagagttt aaagagaaaa gggaaaaaag aaagaaaaag taagcagggc
2341 tggcttcgcc cgcgttctcg tcgtcggatc aaggagcgcg gcggcgtttt ggacccgcgc
2401 tcccatcccc caccttcccg ggccggggac ccactctgcc cagccggagg gacgcggagg
2461 aggaagaggg tagacagggg cgacctgtga ttgttgttat tgatgttgtt gttgatggca
2521 aaaaaaaaaa agcgacttcg agtttgctcc cctttgcttg aagagacccc ctcccccttc
2581 caacgagctt ccggacttgt ctgcaccccc agcaagaagg cgagttagtt ttctagagac
2641 ttgaaggagt ctcccccttc ctgcatcacc accttggttt tgttttattt tgcttcttgg
2701 tcaagaaagg aggggagaac ccagcgcacc cctccccccc tttttttaaa cgcgtgatga
2761 agacagaagg ctccggggtg acgaatttgg ccgatggcag atgttttggg ggaacgccgg
2821 gactgagaga ctccacgcag gcgaattccc gtttggggct tttttttcct ccctcttttc
2881 cccttgcccc ctctgcagcc ggaggaggag atgttgaggg gaggaggcca gccagtgtga
2941 ccggcgctag gaaatgaccc gagaaccccg ttggaagcgc agcagcggga gctaggggcg
3001 ggggcggagg aggacacgaa ctggaagggg gttcacggtc aaactgaaat ggatttgcac
3061 gttggggagc tggcggcggc ggctgctggg cctccgcctt cttttctacg tgaaatcagt
3121 gaggtgagac ttcccagacc ccggaggcgt ggaggagagg agactgtttg atgtggtaca
3181 ggggcagtca gtggagggcg agtggtttcg gaaaaaaaaa aagaaaaaaa gaaaaaaaaa
3241 gaaaaaaaaa agattttttt cttctcttaa toggaatcgt gatggtgttg gattatttca
3301 atggtggggt taatatagca tgttatcctg tctatctttt aaagatttct gtataagact
3361 gttgagcagt ttttaaaata gtgtaggata atataaaaag cagatagatg gcgctatgtt
3421 tgattcctac aacgaaatta tcaccagctt tttttcattc ttaactcttt aaaggattca
3481 aacgcaactc aaatctgtgc tggactttaa aaaaacaatt caggaccaaa ttttttctca
3541 gtgtgtgtgt ttattcctta taggtgtaaa tgagaagacg tgtttttttc cttcaccgat
3601 gctccatcct cgtatttctt tttccttgta aatgtaatca gatgccattt tatatgtgga
3661 cgtatttata ctggccaaac atattttttc ttttgtccct ttttttcttt cctttctttt
3721 tacttccttt atttetttat tccttccttt tccttttttt cttttttttt tctttttttt
3781 tttttttttt tggtagttgt tgttacccac gccattttac gtctccttca ctgaagggct
3841 agagttttaa cttttaattt tttatattta aatgtagact tttgacactt ttaaaaaaca
3901 aaaaaagaca agagagatga aaacgtttga ttattttctc agtgtatttt tgtaaaaaat
3961 atataaaggg ggtgttaatc ggtgtaaatc gctgtttgga tttcctgatt ttataacagg
4021 gcggctggtt aatatctcac acagtttaaa aaatcagccc ctaatttctc catgtttaca
4081 cttcaatctg caggcttctt aaagtgacag tatcccttaa cctgccacca gtgtccaccc
4141 tccggccccc gtcttgtaaa aaggggagga gaattagcca aacactgtaa gcttttaaga
4201 aaaacaaagt tttaaacgaa atactgctct gtccagaggc tttaaaactg gtgcaattac
4261 agcaaaaagg gattctgtag ctttaacttg taaaccacat cttttttgca ctttttttat
4321 aagcaaaaac gtgccgttta aaccactgga tctatctaaa tgccgatttg agttcgcgac
4381 actatgtact gcgtttttca ttcttgtatt tgactattta atcctttcta cttgtcgcta
4441 aatataattg ttttagtctt atggcatgat gatagcatat gtgttcaggt ttatagctgt
4501 tgtgtttaaa aattgaaaaa agtggaaaac atctttgtac atttaagtct gtattataat
4561 aagcaaaaag attgtgtgta tgtatgttta atataacatg acaggcacta ggacgtctgc
4621 ctttttaagg cagttcogtt aagggttttt gtttttaaac ttttttttgc catccatoct
4681 gtgcaatatg ccgtgtagaa tatttgtctt aaaattcaag gccacaaaaa caatgtttgg
4741 gggaaaaaaa agaaaaaatc atgccagcta atcatgtcaa gttcactgcc tgtcagattg
4801 ttgatatata ccttctgtaa ataacttttt ttgagaagga aataaaatca gctggaactg
4861 aaccctaaa
```

One example of an amiR Sox4 molecule is as follows:

(SEQ ID NO: 3)
AGGGCTCTGCGTTTGCTCCAGGTAGTCCGCTGCTCCCTTGGGCCT
GGGCCCACTGACAGCCCTGGTGCCTCTGGCCGGCTGCACACCTCC
TGGCGGGCAGCTGTGTCGTCTTCGAACTCGTCGTCGTGTTCTGGC
AATACCTGCGACGACGTCTCCGAAGACGACACGGAGGCCTGCCCT
GACTGCCCACGGTGCCGTGGCCAAAGAGGATCTAAGGGCACCGCT
GAGGGCCTACCTAACCATCGTGGGGAATAAGGACAGTGTCACCC.
The guide strand of smiR Sox4 is underlined.

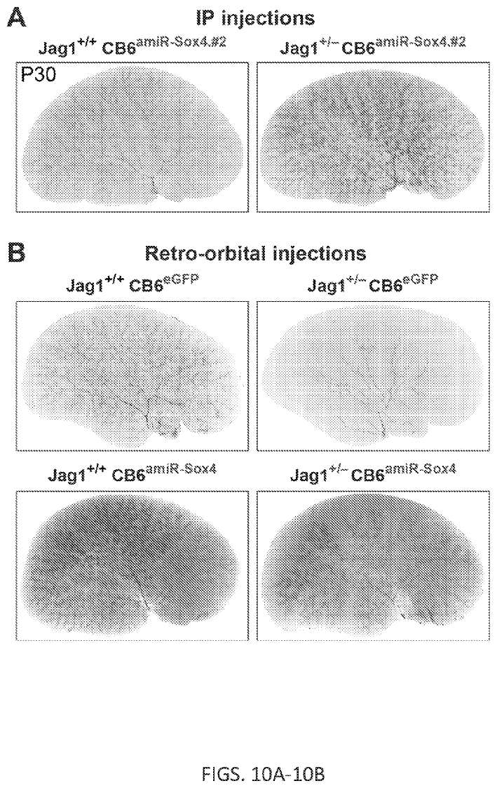

Another example of a amiR-Sox4 molecule is as follows: (FIG. 10):

(SEQ ID NO: 4)
AGGGCTCTGCGTTTGCTCCAGGTAGTCCGCTGCTCCCTTGGGCCT
GGGCCCACTGACAGCCCTGGTGCCTCTGGCCGGCTGCACACCTCC
TGGCGGGCAGCTGTGTAAACCTGAACACATATGCTATGTTCTGGC
AATACCTGTAGCATATCAGCTCAGGTTTACACGGAGGCCTGCCCT
GACTGCCCACGGTGCCGTGGCCAAAGAGGATCTAAGGGCACCGCT
GAGGGCCTACCTAACCATCGTGGGGAATAAGGACAGTGTCACCC.
The guide strand of this amiR Sox4 is
underlined.

Alternatively, or in addition, a corresponding SOX4 gene product may be blocked from being produced, or reduced in its level, and/or may have its activity modified (Such as with a small molecule or antibody), and a representative example of a SOX4 protein is at GenBank® Accession No. NP_003098, which is as follows:

(SEQ ID NO: 2)
MVQQTNNAENTEALLAGESSDSGAGLELGIASSPTPGSTASTGGK
ADDPSWCKTPSGHIKRPMNAFMVWSQIERRKIMEQSPDMHNAEIS
KRLGKRWKLLKDSDKIPFIREAERLRLKHMADYPDYKYRPRKKVK
SGNANSSSSAAASSKPGEKGDKVGGSGGGGHGGGGGGGSSNAGGG
GGGASGGGANSKPAQKKSCGSKVAGGAGGGVSKPHAKLILAGGGG
GGKAAAAAAASFAAEQAGAAALLPLGAAADHHSLYKARTPSASAS
ASSAASASAALAAPGKHLAEKKVKRVYLFGGLGTSSSPVGGVGAG
ADPSDPLGLYEEEGAGCSPDAPSLSGRSSAASSPAAGRSPADHRG
YASLRAASPAPSSAPSHASSSASSHSSSSSSSGSSSSDDEFEDDL
LDLNPSSNFESMSLGSFSSSSALDRDLDFNFEPGSGSHFEFPDYC
TPEVSEMISGDWLESSISNLVFTY

Particularly, an inhibitory oligonucleotide may be capable of decreasing the expression of SOX4 by at least 10%, 20%, 30%, or 40%, more particularly by at least 50%, 60%, or 70%, and most particularly by at least 75%, 80%, 90%, 95%, 99%, or 100% or any range or value in between the foregoing.

In further embodiments, there are synthetic oligonucleotides that are SOX4 inhibitors. An inhibitor may be between 17 to 25 nucleotides in length and comprises a 5′ to 3′ sequence that is at least 90% complementary to the 5′ to 3′ sequence of a mature SOX4 mRNA. In certain embodiments, an inhibitor molecule is 17, 18, 19, 20, 21, 22, 23, 24, or 25 nucleotides in length, or any range derivable therein. Moreover, an inhibitor molecule has a sequence (from 5′ to 3′) that is or is at least 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein, to the 5′ to 3′ sequence of a mature SOX4 mRNA, particularly a mature, naturally occurring mRNA. One of skill in the art could use a portion of the probe sequence that is complementary to the sequence of a mature mRNA as the sequence for an mRNA inhibitor. Moreover, that portion of the probe sequence can be altered so that it is still 90% complementary to the sequence of a mature mRNA.

In some embodiments, the inhibitory oligonucleotide is an analog and may include modifications, particularly modifications that increase nuclease resistance, improve binding affinity, and/or improve binding specificity. For example, when the sugar portion of a nucleoside or nucleotide is replaced by a carbocyclic moiety, it is no longer a sugar. Moreover, when other substitutions, such a substitution for the inter-sugar phosphodiester linkage are made, the resulting material is no longer a true species. All such compounds are considered to be analogs. Throughout this specification, reference to the sugar portion of a nucleic acid species shall be understood to refer to either a true sugar or to a species taking the structural place of the sugar of wild type nucleic acids. Moreover, reference to inter-sugar linkages shall be taken to include moieties serving to join the sugar or sugar analog portions in the fashion of wild type nucleic acids.

The present disclosure concerns modified oligonucleotides, i.e., oligonucleotide analogs or oligonucleosides, and methods for effecting the modifications. These modified oligonucleotides and oligonucleotide analogs may exhibit increased chemical and/or enzymatic stability relative to their naturally occurring counterparts. Extracellular and intracellular nucleases generally do not recognize and therefore do not bind to the backbone-modified compounds. When present as the protonated acid form, the lack of a negatively charged backbone may facilitate cellular penetration.

The modified internucleoside linkages are intended to replace naturally-occurring phosphodiester-5′-methylene linkages with four atom linking groups to confer nuclease resistance and enhanced cellular uptake to the resulting compound.

Modifications may be achieved using solid supports which may be manually manipulated or used in conjunction with a DNA synthesizer using methodology commonly known to those skilled in DNA synthesizer art. Generally, the procedure involves functionalizing the sugar moieties of two nucleosides which will be adjacent to one another in the selected sequence. In a 5′ to 3′ sense, an “upstream” synthon such as structure H is modified at its terminal 3′ site, while a “downstream” synthon such as structure H1 is modified at its terminal 5′ site.

Oligonucleosides linked by hydrazines, hydroxylarnines, and other linking groups can be protected by a dimethoxytrityl group at the 5′-hydroxyl and activated for coupling at the 3′-hydroxyl with cyanoethyldiisopropyl-phosphite moieties. These compounds can be inserted into any desired sequence by standard, solid phase, automated DNA synthesis techniques. One of the most popular processes is the phosphoramidite technique. Oligonucleotides containing a uniform backbone linkage can be synthesized by use of CPG-solid support and standard nucleic acid synthesizing machines such as Applied Biosystems Inc. 380B and 394 and Milligen/Biosearch 7500 and 8800s. The initial nucleotide (number 1 at the 3′-terminus) is attached to a solid support such as controlled pore glass. In sequence specific order, each new nucleotide is attached either by manual manipulation or by the automated synthesizer system.

Free amino groups can be alkylated with, for example, acetone and sodium cyanoboro hydride in acetic acid. The alkylation step can be used to introduce other, useful, functional molecules on the macromolecule. Such useful functional molecules include but are not limited to reporter molecules, RNA cleaving groups, groups for improving the pharmacokinetic properties of an oligonucleotide, and groups for improving the pharmacodynamic properties of an oligonucleotide. Such molecules can be attached to or conjugated to the macromolecule via attachment to the nitrogen atom in the backbone linkage. Alternatively, such molecules can be attached to pendent groups extending from a hydroxyl group of the sugar moiety of one or more of the nucleotides. Examples of such other useful functional groups are provided by WO1993007883, which is herein incorporated by reference, and in other of the above-referenced patent applications.

Solid supports may include any of those known in the art for polynucleotide synthesis, including controlled pore glass (CPG), oxalyl controlled pore glass, TentaGel Support—an aminopolyethyleneglycol derivatized support or Poros—a copolymer of polystyrene/divinylbenzene. Attachment and cleavage of nucleotides and oligonucleotides can be effected via standard procedures. As used herein, the term solid support further includes any linkers (e.g., long chain alkyl amines and succinyl residues) used to bind a growing oligonucleoside to a stationary phase such as CPG. In some embodiments, the oligonucleotide may be further defined as having one or more locked nucleotides, ethylene bridged nucleotides, peptide nucleic acids, or a 5′(E)-vinyl-phosphonate (VP) modification. In some embodiments, the oligonucleotide has one or more phosphorothioated DNA or RNA bases.

### D. Vectors

In some aspects, contemplated are expression vectors comprising a nucleic acid molecule encoding an inhibitory nucleic acid or polypeptide of a desired sequence or a portion thereof that targets SOX4. In some aspects, expression vectors comprising nucleic acid molecules may encode inhibitory nucleic acid such as those that bind a part of a SOX4 nucleic acid such as the one represented by SEQ ID NO:1. In addition to control sequences that govern transcription and translation, vectors and expression vectors may contain nucleic acid sequences that serve other functions as well.

To express the desired inhibitory nucleic acid or gene product, DNAs encoding the SOX4 nucleic acid of interest are inserted into expression vectors such that the gene area is operatively linked to transcriptional and translational control sequences. In some cases, expression vectors contain sequences for plasmid or virus maintenance and for cloning and expression of exogenous nucleotide sequences. Such sequences, collectively referred to as “flanking sequences” typically include one or more of the following operatively linked nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Such sequences and methods of using the same are well known in the art.

The vector on which the inhibitory nucleic acid resides may be of any kind, including viral or non-viral (plasmid, transposon, etc.). Viral vectors can bring genetic material into cells. The principle is to use the molecular mechanism of viruses to transmit their genomes into other cells for infection. Viral vectors can also be referred to as vectors, vector virus particles, or vector particles. Examples of viral vectors include, but are not limited to: adeno-associated virus, retrovirus, adenovirus, herpes simplex virus, vaccinia virus, baculovirus, or lentivirus.

Adeno-associated virus (adeno-associated virus, AAV), also known as adeno-associated virus, belongs to the genus of dependent viruses in the Parvoviridae family, and is the simplest type of single-stranded DNA-deficient virus found so far. Recombinant AAV vectors have been successfully used for the transduction of marker genes and genes involved in human diseases in vitro and in vivo. Certain AAV vectors have been developed, which can effectively bind large payloads (up to 8-9 kb). Any serotype can be utilized, including AAV1, AAV2, AAV3, AAV4, AAV5, AAV6, AAV7, AAV8, AAV9, AAV10, or AAV11, for example. In particular embodiments, AAV8 is utilized as the vector to deliver inhibitory nucleic acids to individuals having bile duct paucity.

The retroviral vector can be derived or capable of being derived from any suitable retrovirus. A large number of different retroviruses have been identified. Examples include but are not limited to: murine leukemia virus (MLV), human T-cell leukemia virus (HTLV), mouse breast tumor virus (MMTV), Rous sarcoma virus (RSV), Fujinami sarcoma virus (FuSV), Moloney Murine Leukemia Virus (Mo MLV), FBR Murine Osteosarcoma Virus (FBR MSV), Moloney Murine Sarcoma Virus (Mo-MSV), Abelson Murine Leukemia Virus (A-MLV), Avian Myeloma Virus-29 (MC29) And Avian Polycythemia Virus (AEV).

Adenovirus is a double-stranded linear DNA virus, which does not replicate through RNA intermediates. Adenovirus is a double-stranded DNA non-enveloped virus, which can transduce a wide range of human and non-human cell types in vivo, in vitro and in vitro. These cells include airway epithelial cells, hepatocytes, muscle cells, cardiomyocytes, synovial cells, primary breast epithelial cells, and terminally differentiated cells (such as neurons) after mitosis. Adenovirus has been used as a vector for gene therapy and heterologous gene expression. The large (36 kb) genome can accommodate up to 8 kb of foreign inserted DNA, and can replicate efficiently in complementary cell lines to produce very high titer of up to 1012 transduction units per milliliter. Adenovirus is therefore one of the best systems for studying gene expression in primary non-replicating cells. The expression of viral genes or foreign genes from the adenoviral genome does not require replicating cells. Adenovirus vectors enter cells through receptor-mediated endocytosis. Once inside the cell, adenovirus vectors rarely integrate into the host chromosome. Instead, they exist as episomes (independent of the host genome) as a linear genome in the host cell nucleus.

Herpes simplex virus (HSV) is an enveloped double-stranded DNA virus that naturally infects neurons. It can accommodate large segments of foreign DNA and has been adopted as a carrier for gene delivery to neurons. The use of HSV in the treatment process requires attenuation of the virus strains, so that they cannot establish a lysis cycle. In particular, if the HSV vector is used for gene therapy in humans, it is preferable to insert the polynucleotide into the essential gene. This is because if the viral vector encounters a wild-type virus, the heterologous gene can be transferred to the wild-type virus through recombination. However, if the recombinant virus is constructed in a way to prevent its replication, this can be achieved by inserting oligonucleotides into viral genes necessary for replication.

The viral vector of the present disclosure may be a vaccinia virus vector, such as MVA or NYVAC. Alternatives to vaccinia vectors include, for example, fowlpox or canarypox (avipox) vectors called ALVAC, and strains derived therefrom, which can infect and express recombinant proteins in human cells but cannot replicate. It should be understood that part of the viral genome can remain intact after the insertion of the recombinant gene. This means that viral vectors can retain the concept of the ability to infect cells and subsequently express additional genes that support their replication and may promote the lysis and death of infected cells.

Lentiviruses are part of a larger group of retroviruses. Can be divided into primate and non-primate groups. Examples of primate lentiviruses include, but are not limited to: human immunodeficiency virus (HIV), the pathogen of human autoimmune deficiency syndrome (AIDS), and simian immunodeficiency virus (SIV). The non-primate lentivirus population includes the prototype “lentivirus” visna/maedi virus (VMV), as well as the related goat arthritis-encephalitis virus (CAEV), equine infectious anemia virus (EIAV), and feline immunodeficiency virus (FIV) and Bovine Immunodeficiency Virus (BTV).

### E. Antibodies

In particular embodiments, an individual with bile duct paucity, including who may have AGLS, is administered an effective amount of one or more anti-SOX4 antibodies of any kind. The antibodies may be commercially obtained or may be generated by the user or otherwise obtained by the user. A SOX4 antibody may be administered to an individual systemically or locally, such as through the intrahepatic vein, as an example.

The term “antibody” refers to an intact immunoglobulin of any isotype, or a fragment thereof that can compete with the intact antibody for specific binding to SOX4 protein, and includes chimeric, humanized, fully human, and bispecific antibodies. As used herein, the terms “antibody” or “immunoglobulin” are used interchangeably and refer to any of several classes of structurally related proteins that function as part of the immune response of an animal, including IgG, IgD, IgE, IgA, IgM, and related proteins, as well as polypeptides comprising antibody CDR domains that retain antigen-binding activity.

The term “antigen” refers to a molecule or a portion of a molecule capable of being bound by a selective binding agent, such as an antibody. An antigen may possess one or more epitopes that are capable of interacting with different antibodies.

The term “epitope” includes any region or portion of molecule capable eliciting an immune response by binding to an immunoglobulin or to a T-cell receptor. Epitope determinants may include chemically active surface groups such as amino acids, sugar side chains, phosphoryl or sulfonyl groups, and may have specific three-dimensional structural characteristics and/or specific charge characteristics. Generally, antibodies specific for a particular target antigen will preferentially recognize an epitope on the target antigen within a complex mixture.

The epitope regions of a given polypeptide can be identified using many different epitope mapping techniques are well known in the art, including: x-ray crystallography, nuclear magnetic resonance spectroscopy, site-directed mutagenesis mapping, protein display arrays, see, e.g., Epitope Mapping Protocols, (Johan Rockberg and Johan Nilvebrant, Ed., 2018) Humana Press, New York, N.Y. Such techniques are known in the art and described in, e.g., U.S. Pat. No. 4,708,871; Geysen et al. Proc. Natl. Acad. Sci. USA 81:3998-4002 (1984); Geysen et al. Proc. Natl. Acad. Sci. USA 82:178-182 (1985); Geysen et al. Molec. Immunol. 23:709-715 (1986). Additionally, antigenic regions of proteins can also be predicted and identified using standard antigenicity and hydropathy plots.

The term “immunogenic sequence” means a molecule that includes an amino acid sequence of at least one epitope such that the molecule is capable of stimulating the production of antibodies in an appropriate host. The term “immunogenic composition” means a composition that comprises at least one immunogenic molecule (e.g., an antigen or carbohydrate).

An intact antibody is generally composed of two full-length heavy chains and two full-length light chains, but in some instances may include fewer chains, such as antibodies naturally occurring in camelids that may comprise only heavy chains. Antibodies as disclosed herein may be derived solely from a single source or may be “chimeric,” that is, different portions of the antibody may be derived from two different antibodies. For example, the variable or CDR regions may be derived from a rat or murine source, while the constant region is derived from a different animal source, such as a human. The antibodies or binding fragments may be produced in hybridomas, by recombinant DNA techniques, or by enzymatic or chemical cleavage of intact antibodies. Unless otherwise indicated, the term “antibody” includes derivatives, variants, fragments, and muteins thereof, examples of which are described below (Sela-Culang et al., Front Immunol. 2013; 4: 302; 2013).

An intact antibody is generally composed of two full-length heavy chains and two full-length light chains, but in some instances may include fewer chains, such as antibodies naturally occurring in camelids that may comprise only heavy chains. Antibodies as disclosed herein may be derived solely from a single source or may be “chimeric,” that is, different portions of the antibody may be derived from two different antibodies. For example, the variable or CDR regions may be derived from a rat or murine source, while the constant region is derived from a different animal source, such as a human. The antibodies or binding fragments may be produced in hybridomas, by recombinant DNA techniques, or by enzymatic or chemical cleavage of intact antibodies. Unless otherwise indicated, the term “antibody” includes derivatives, variants, fragments, and muteins thereof, examples of which are described below (Sela-Culang et al. Front Immunol. 2013; 4: 302; 2013)

The isotype of an antibody can be IgM, IgD, IgG, IgA, or IgE and is defined by the heavy chains present of which there are five classifications: mu (μ), delta (δ), gamma (γ), alpha (α), or epsilon (ε) chains, respectively. IgG has several subtypes, including, but not limited to, IgG1, IgG2, IgG3, and IgG4. IgM subtypes include IgM1 and IgM2. IgA subtypes include IgA1 and IgA2.

Antibodies can be whole immunoglobulins of any isotype or classification, chimeric antibodies, or hybrid antibodies with specificity to two or more antigens. They may also be fragments (e.g., F(ab′)2, Fab′, Fab, Fv, and the like), including hybrid fragments. An immunoglobulin also includes natural, synthetic, or genetically engineered proteins that act like an antibody by binding to specific antigens to form a complex. The term antibody includes genetically engineered or otherwise modified forms of immunoglobulins, such as the following:

In certain aspects, the antigen-binding domain may be multispecific or heterospecific by multimerizing with VH and VL region pairs that bind a different antigen. For example, the antibody may bind to, or interact with, (a) a cell surface antigen, (b) an Fc receptor on the surface of an effector cell, or (c) at least one other component. Accordingly, aspects may include, but are not limited to, bispecific, trispecific, tetraspecific, and other multispecific antibodies or antigen-binding fragments thereof that are directed to epitopes and to other targets, such as Fe receptors on effector cells.

In some embodiments, multispecific antibodies can be used and directly linked via a short flexible polypeptide chain, using routine methods known in the art. One such example is diabodies that are bivalent, bispecific antibodies in which the VH and VL domains are expressed on a single polypeptide chain, and utilize a linker that is too short to allow for pairing between domains on the same chain, thereby forcing the domains to pair with complementary domains of another chain creating two antigen binding sites. The linker functionality is applicable for embodiments of triabodies, tetrabodies, and higher order antibody multimers. (see, e.g., Hollinger et al., Proc Natl. Acad. Sci. USA 90:6444-6448 (1993); Polijak et al., Structure 2:1121-1123 (1994); Todorovska et al., J. Immunol. Methods 248:47-66 (2001)).

Polyclonal antibody preparations may be utilized and typically include different antibodies against different determinants (epitopes). In order to produce polyclonal antibodies, a host, such as a rabbit or goat, is immunized with the antigen or antigen fragment, generally with an adjuvant and, if necessary, coupled to a carrier. Antibodies to the antigen are subsequently collected from the sera of the host. The polyclonal antibody can be affinity purified against the antigen rendering it monospecific.

Monoclonal antibodies or “mAb” may be utilized and refer to an antibody obtained from a population of homogeneous antibodies from an exclusive parental cell, e.g., the population is identical except for naturally occurring mutations that may be present in minor amounts. Each monoclonal antibody is directed against a single antigenic determinant.

Certain aspects relate to antibody fragments, such as antibody fragments that bind to and/or neutralize inflammatory mediators. The term functional antibody fragment includes antigen-binding fragments of an antibody that retain the ability to specifically bind to an antigen. These fragments are constituted of various arrangements of the variable region heavy chain (VH) and/or light chain (VL); and in some embodiments, include constant region heavy chain 1 (CH1) and light chain (CL). In some embodiments, they lack the Fc region constituted of heavy chain 2 (CH2) and 3 (CH3) domains. Embodiments of antigen binding fragments and the modifications thereof may include: (i) the Fab fragment type constituted with the VL, VH, CL, and CH1 domains; (ii) the Fd fragment type constituted with the VH and CH1 domains; (iii) the Fv fragment type constituted with the VH and VL domains; (iv) the single domain fragment type, dAb, (Ward, 1989; McCafferty et al., 1990; Holt et al., 2003) constituted with a single VH or VL domain; (v) isolated complementarity determining region (CDR) regions. Such terms are described, for example, in Harlow and Lane, Antibodies: A Laboratory Manual, Cold Spring Harbor Laboratory, NY (1989); Molec. Biology and Biotechnology: A Comprehensive Desk Reference (Myers, R. A. (ed.), New York: VCH Publisher, Inc.); Huston et al., Cell Biophysics, 22:189-224 (1993); Pluckthun and Skerra, Meth. Enzymol., 178:497-515 (1989) and in Day, E. D., Advanced Immunochemistry, 2d ed., Wiley-Liss, Inc. New York, N.Y. (1990); Antibodies, 4:259-277 (2015). The citations in this paragraph are all incorporated by reference.

Antigen-binding fragments also include fragments of an antibody that retain exactly, at least, or at most 1, 2, or 3 complementarity determining regions (CDRs) from a light chain variable region. Fusions of CDR-containing sequences to an Fe region (or a CH2 or CH3 region thereof) are included within the scope of this definition including, for example, scFv fused, directly or indirectly, to an Fe region are included herein.

The term Fab fragment means a monovalent antigen-binding fragment of an antibody containing the VL, VH, CL and CH1 domains. The term Fab′ fragment means a monovalent antigen-binding fragment of a monoclonal antibody that is larger than a Fab fragment. For example, a Fab′ fragment includes the VL, VH, CL and CH1 domains and all or part of the hinge region. The term F(ab′)2 fragment means a bivalent antigen-binding fragment of a monoclonal antibody comprising two Fab′ fragments linked by a disulfide bridge at the hinge region. An F(ab′)2 fragment includes, for example, all or part of the two VH and VL domains, and can further include all or part of the two CL and CH1 domains.

The term Fv fragment means a monovalent antigen-binding fragment of a monoclonal antibody, including all or part of the VL and VH, and absent of the CL and CH1 domains. The VL and VH include, for example, the CDRs. Single-chain antibodies (sFv or scFv) are Fv molecules in which the VL and VH regions have been connected by a flexible linker to form a single polypeptide chain, which forms an antigen-binding fragment. Single chain antibodies are discussed in detail in International Patent Application Publication No. WO 88/01649 and U.S. Pat. Nos. 4,946,778 and 5,260,203, the disclosures of which are herein incorporated by reference. The term (scFv)2 means bivalent or bispecific sFv polypeptide chains that include oligomerization domains at their C-termini, separated from the sFv by a hinge region (Pack et al. 1992). The oligomerization domain comprises self-associating a-helices, e.g., leucine zippers, which can be further stabilized by additional disulfide bonds. (scFv)2 fragments are also known as “miniantibodies” or “minibodies.”

A single domain antibody is an antigen-binding fragment containing only a VH or the VL domain. In some instances, two or more VH regions are covalently joined with a peptide linker to create a bivalent domain antibody. The two VH regions of a bivalent domain antibody may target the same or different antigens.

An Fc region contains two heavy chain fragments comprising the CH2 and CH3 domains of an antibody. The two heavy chain fragments are held together by two or more disulfide bonds and by hydrophobic interactions of the CH3 domains. The term “Fe polypeptide” as used herein includes native and mutein forms of polypeptides derived from the Fc region of an antibody. Truncated forms of such polypeptides containing the hinge region that promotes dimerization are included.

### V. Administration of Therapeutic Compositions

The therapy provided herein may comprise administration of a combination of therapeutic agents, such as one or more agents that inhibit expression and/or activity of SOX4 gene or SOX4 protein.

In some embodiments, pharmaceutical compositions comprising one or more agents that inhibit SOX4 expression and or SOX4 activity are administered to a subject. Different aspects may involve administering an effective amount of a composition to a subject. In some embodiments, a therapeutic composition capable of binding to SOX4 gene or protein may be administered to the subject to protect against or treat a condition (e.g., bile duct paucity, such as ALGS). Additionally, such compositions can be administered in combination with an additional therapeutic agent (e.g., ursodiol, cholestyramine, Maralixibat, a combination thereof, and/or one or more treatments for non-liver related aspects of ALGS, etc.). Such compositions will generally be dissolved or dispersed in a pharmaceutically acceptable carrier or aqueous medium.

The therapeutic agent(s) of the disclosure may be administered by any suitable route of administration. In some embodiments, the therapy is administered through the hepatic vein, intrahepatic injection, intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of bile duct paucity to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. In specific embodiments, the quantity is that which results in knockdown of about 50% to about 70% of expression compared to that in the absence of knockdown. The amount of knockdown may be about 50, 51, 52, 53, 54, 55, 56, 57, 58, 59, 60, 61, 62, 63, 64, 65, 66, 67, 68, 69, or 70% compared to that in the absence of knockdown. The range of knockdown may be 50-70, 50-65, 50-60, 50-55, 55-70, 55-65, 55-60, 60-70, 60-65, or 65-70% compared to that in the absence of knockdown.

In specific embodiments, the amount of vector compositions to administer to a human patient is the amount that is approximately equivalent to 2×10¹¹ genomes per animal in mice.

An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In some embodiments, the vector is administered once into an individual. In certain embodiments, it will be desirable to have multiple administrations of the composition, e.g., 2, 3, 4, 5, 6 or more administrations. The administrations can be at 1, 2, 3, 4, 5, 6, 7, 8, to 5, 6, 7, 8, 9, 10, 11, or 12 day or week intervals, including all ranges there between.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The active compounds can be formulated for parenteral administration, e.g., formulated for injection via the intravenous, intramuscular, subcutaneous, intrahepatic, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to oral, or intravenous administration. Alternatively, administration may be by orthotopic, intradermal, subcutaneous, intramuscular, intraperitoneal, intrahepatic, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective. The formulations are easily administered in a variety of dosage forms, such as the type of injectable solutions described above.

### VI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### An Aav-Based Treatment for Alagille Syndrome

It has previously been reported that removing one or both copies of Sox9 with Albumin-Cre driver (20) results in a dosage-sensitive worsening of most phenotypes in Jag1^{+/-} mouse livers (19). Sox4 is a related transcription factor which has been reported to have an overlapping role with Sox9 in biliary development (22). Specifically, it was shown that although conditional loss of Sox9 or Sox4 in the liver leads to mild and mostly reversible defects in biliary development, conditional loss of both genes leads to rather severe BD abnormalities (22). It was therefore predicted that removing one copy of Sox4 would worsen the Jag1^{+/-} phenotypes similar to removing one copy of Sox9. In order to test this, one copy of Sox4 was removed with the same Albumin-Cre driver in Jag1^{+/-} animals using a mouse line with loxP sites flanking the sole exon in the Sox4 gene (23), generating Jag1^{+/-}; Sox4^{+/flox}; Albumin-Cre mice (hereafter called Jag1^{+/-}; Sox4^{+/Δ}).

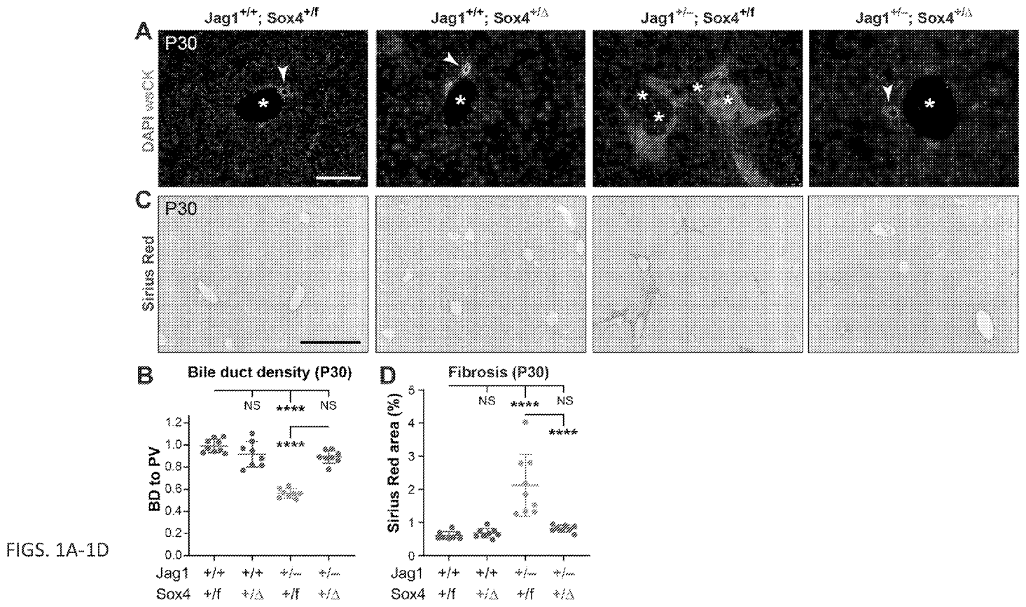

At 30 days of age (P30), wild-type animals and those lacking one copy of Sox4 in the liver (Jag1^{+/-}; Sox4^{+/Δ}) exhibit patent BDs around PVs (FIG. 1A). In agreement with previous reports (18, 19), Jag1^{+/-} animals show a statistically significant reduction in the BD to PV ratio (0.56 in Jag1 Jiversus 0.99 in Jag1^{+/-}; Sox4^{+/f} and 0.92 in Jag1^{+/-}; Sox4^{+/Δ} livers), accompanied by ductular reactions and periportal hypercellularity (FIGS. 1A, 1C), which are caused by accumulation of cytokeratin+reactive biliary cells and infiltration of inflammatory cells in response to BD paucity and likely excess bile buildup. Unexpectedly, Jag1^{+/-}; Sox4^{+/Δ} livers show an improvement in the formation of bile ducts, as well as the BD to PV ratio (FIGS. 1A, 1C). Removal of one copy of Sox4 also eliminates the ductular reaction and periportal hypercellularity (FIG. 1A). Sirius Red staining of Jag1^{+/-} livers demonstrates moderate fibrosis, which often bridges between portal veins and extends into the parenchyma of the liver (FIG. 1C; (18, 19)). This bridging indicates moderate, or grade 3 fibrosis (24). On average, Sirius Red+ area constitutes 2.13% of the total liver area in P30 Jag1^{+/-} mice (FIG. 1D). However, removal of one copy of Sox4 in the livers of Jag1^{+/-} mice significantly reduces the liver fibrosis in these animals with an average Sirius Red+ area of 0.82%, which is not statistically different than control livers (FIGS. 1B, 1D). These observations indicate that reducing Sox4 levels in the liver significantly improves biliary development and reduces the liver damage response in Jag1^{+/-} animals.

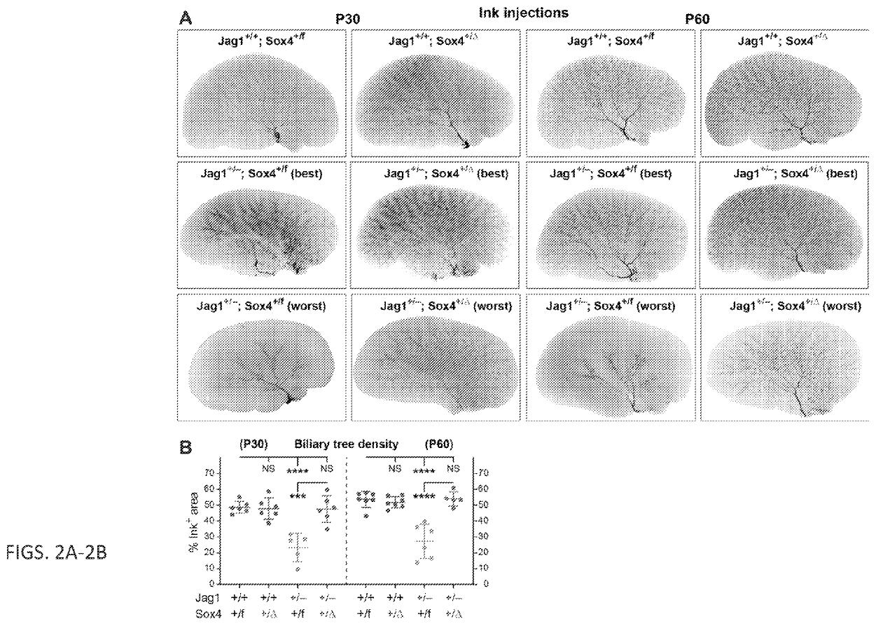

Next, retrograde biliary ink injection was performed to visualize the biliary system in three dimensions, as described previously (25). Control (Jag1^{+/-}; Sox4^{+/f} and Jag1^{+/-}; Sox4^{+/Δ}) animals show dense, robust biliary trees which extend to the periphery of the organ at both P30 and P60 (FIG. 2A). This illustrates the ability to clear bile from every part of the liver. In agreement with a previous report (19), Jag1^{+/-} animals show impaired biliary tree formation with some degree of variability (FIG. 2A). Specifically, the Jag1^{+/-} biliary trees are sparse and fail to reach the liver periphery, leaving areas of the liver not accessed by ducts and likely unable to drain bile. Jag1^{+/-}; Sox4^{+/Δ} livers show robust and dense biliary trees, which extend closer to the periphery of the organ compared to Jag1^{+/-} livers, although some gaps in the peripheral-most areas of the liver are still seen (FIG. 2A). Measuring the density of the biliary trees indicates that the average area covered by ink is approximately 48% of the total liver area in control P30 animals (FIG. 2B; 48.75% in Jag1^{+/-}; Sox4^{+/f} and ˜47.92% in Jag1^{+/-}; Sox4^{+/Δ}. In Jag1^{+/-} livers, the biliary tree density is in the range of 9.54%-31.49%, with an average density of 23.29%, which is approximately half of the control (FIG. 2B). However, the average Jag1^{+/-}; Sox4^{+/Δ} ink tree density is 47.58% (34.97%-59.84%), and is not statistically significantly different from the biliary tree density of control animals (FIG. 2B). Two-month old animals were assessed by ink injection to determine if the improvement persists into adulthood. Indeed, the same trend is observed, with the Jag1^{+/-} average density approaching half of the control average density, and the Jag1^{+/-}; Sox4^{+/Δ} density not different from the control density (FIG. 2A, 2B). Importantly, in some Jag1^{+/-}; Sox4^{+/Δ} animals the biliary tree fully extends to the liver periphery and is indistinguishable from control biliary trees (FIG. 2A; Jag1^{+/-}; Sox4^{+/Δ} (best)). These data indicate that loss of one copy of Sox4 in the liver leads to a significant and sustained improvement in the formation of biliary tree in Jag1-haploinsufficient animals.

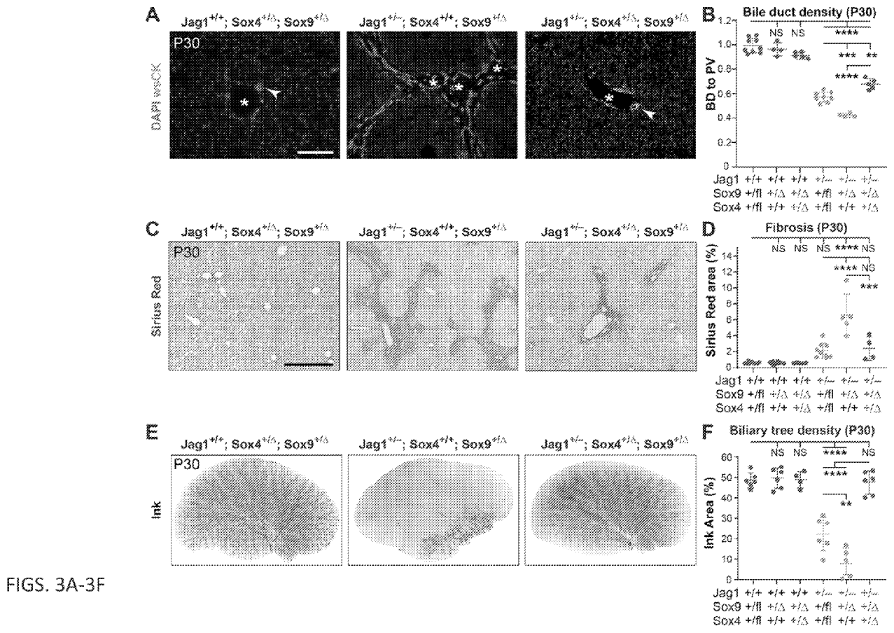

It was next examined whether loss of one copy of Sox4 in the liver also improves the liver phenotypes in Jag1^{+/-}; Sox9^{+/Δ} animals, which represent a model for severe ALGS liver disease (19). As reported previously, removing one copy of Sox9 with Albumin-Cre in Jag1^{+/-} mice leads to worsening of BD formation at P30 compared to Jag1^{+/-} livers (FIG. 3). Specifically, P30 Jag1^{+/-}; Sox9^{+/Δ} animals exhibit larger areas of hypercellularity and ductular reaction with ectopic cytokeratin positive cells and further reduction in the BD to PV ratio to 0.42, compared to Jag1^{+/-} (0.56) and control (0.99) (FIGS. 3A, 3B). When one copy of Sox4 is removed from Jag1^{+/-}; Sox9^{+/Δ} livers, areas of ductular reaction and hypercellularity are greatly reduced, and more patent bile ducts are visible (FIG. 3A). The average BD to PV ratio in Jag1^{+/-}; Sox9^{+/Δ}; Sox4^{+/Δ} animals is approximately 0.68, which constitutes a statistically significant improvement not only compared to Jag1^{+/-}; Sox9^{+/Δ} animals (0.42) but also compared to Jag1^{+/-} animals (0.56) (FIG. 3B). Fibrosis is much more severe in Jag1^{+/-}; Sox9^{+/Δ} animals compared to Jag1^{+/-} animals, with Sirius Red staining covering 6.61% of the total liver area on average (FIGS. 3C, 3D). Moreover, fibrotic areas bridge most portal veins (FIG. 3C), indicating grade 4, or severe fibrosis (24). Removal of one copy of Sox4 in the livers of Jag1^{+/-}; Sox9^{+/Δ} animals reduces the average Sirius Red+ area to 2.43%, near the levels of the Jag1^{+/-} model alone (2.12%) (FIG. 3D). Moreover, bridging is limited to an infrequent occurrence, and when present, fibrotic areas are smaller (FIG. 3C). Finally, in agreement with a previous report, ink injections reveal that in Jag1^{+/-}; Sox9^{+/Δ} animals the biliary tree is severely affected, with ink being confined to a small region at the base of the liver in the most severe cases (FIGS. 3E, 3F). Since ink does not reach most of the liver in these animals, bile must not be able to drain from the vast majority of the liver either and will build up and cause damage. Removal of one copy of Sox4 in these animals results in a greatly improved biliary tree, with a dense and robust tree covering most of the area of the liver (FIG. 3E). Quantification reveals that even the more severely reduced Jag1^{+/-}; Sox9^{+/Δ} biliary tree density observed in Jag1^{+/-}; Sox9^{+/Δ} animals (12.38% on average) can be improved to near control levels (47.65% on average) by removing one copy of Sox4 with Albumin-Cre (FIG. 3F). Together, these data provide compelling evidence that reducing the level of Sox4 in the liver significantly improves the liver phenotypes of both ALGS models used in the studies.

The Albumin-Cre driver starts expressing Cre in hepatoblasts during embryonic development (21). Therefore, animals harboring Albumin-Cre and one copy of the Sox4^{/flox} allele likely lose one copy of Sox4 in many embryonic liver cells before the ALGS models develop BD paucity. In order to assess the ability of reducing Sox4 to improve the ALGS liver phenotypes, it was decided to establish an alternative strategy for knocking down Sox4 after BD paucity has emerged in the models, namely at the first postnatal day (P0-P1) (18, 19).

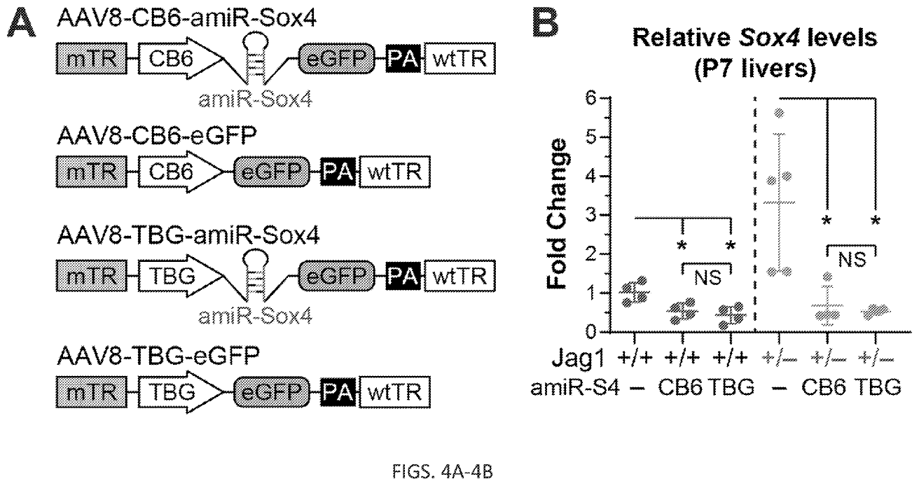

To this end, a ubiquitous promoter-driven artificial microRNA (26) was designed which targets Sox4 mRNA and packaged it into a liver tropic, self-complementary adeno-associated virus serotype 8 (AAV8) (FIG. 4A, AAV8-CB6-amiR-Sox4). Given the genetic rescue of the phenotypes was generated upon removing one copy of Sox4 with Albumin-Cre, a second set of Sox4-targeting AAV8 vectors with the same Sox4 silencing sequence but using the hepatocyte-specific TBG promoter (TBG) (27-30) (FIG. 4A, AAV8-TBG-amiR-Sox4). Control empty vectors were also generated that lack amiR-Sox4 but still drive eGFP (FIG. 4A). A single intraperitoneal (IP) injection of 2×10¹¹ genomes of each vector at P1 led to a significant knockdown of Sox4 in the livers of P7 mice compared to livers from animals injected with a control vector (FIG. 4B). This allowed characterizing a clinically relevant treatment strategy in the mouse models.

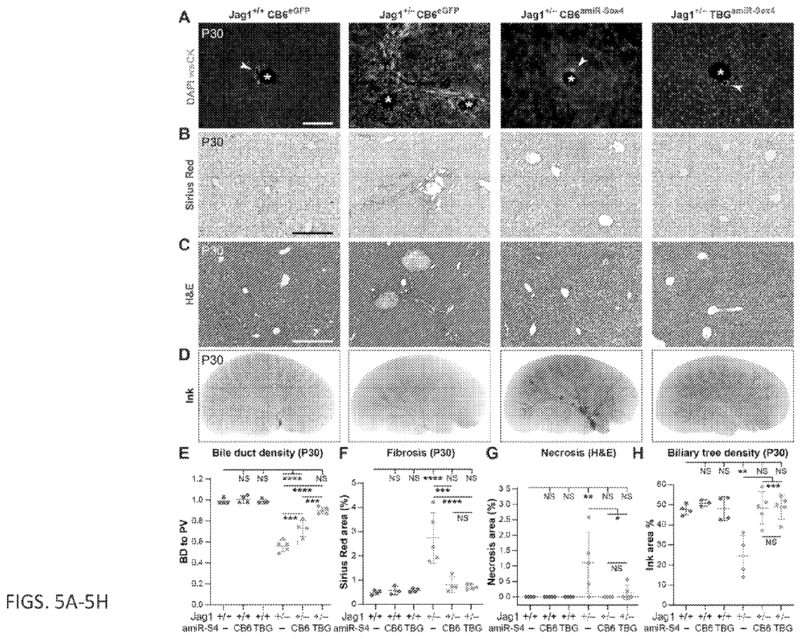

It was first examined whether a single IP injection of 2×10¹¹ genomes of AAV8-CB6-amiR-Sox4 or AAV8-TBG-amiR-Sox4 at P1 can improve the Jag1^{+/-} liver phenotypes at P30. As shown in FIG. 5A, both vectors were able to improve BD formation in Jag1^{+/-} animals, with most areas in the liver not showing ductular reaction and hypercellularity anymore. The BD to PV ratio in these animals was significantly increased compared to Jag1^{+/-} animals injected with the control AAV8-CB6-eGFP vector, with AAV8-TBG-amiR-Sox4 showing a more efficient increase in BD density compared to AAV8-CB6-amiR-Sox4 (FIG. 5E). Upon AAV8-mediated Sox4 knockdown by both vectors, the fibrotic area in Jag1^{+/-} animals is significantly reduced and when present, it rarely if ever bridges between portal veins (FIG. 5B, 5F, not shown). Hematoxylin and Eosin (H&E) staining of liver sections followed by quantification indicates that both vectors rescue the liver necrosis phenotype of Jag1^{+/-} animals as well (FIG. 5C, 5G). Biliary ink injections revealed a strong improvement in biliary tree formation in Jag1^{+/-} livers by both vectors, with greater extension towards the periphery of the organ and fewer gaps in the tree without ductal coverage (FIG. 5D). Quantification of ink density showed that upon AAV8-mediated Sox4 knockdown by both vectors the biliary tree density of Jag1^{+/-} animals is significantly improved compared to control animals (FIG. 5H). It was conclude that a single injection of an AAV8 vector driving Sox4 knockdown ubiquitously or in TBG⁺ cells is able to significantly improve all of the liver phenotypes observed in Jag1^{+/-} animals.

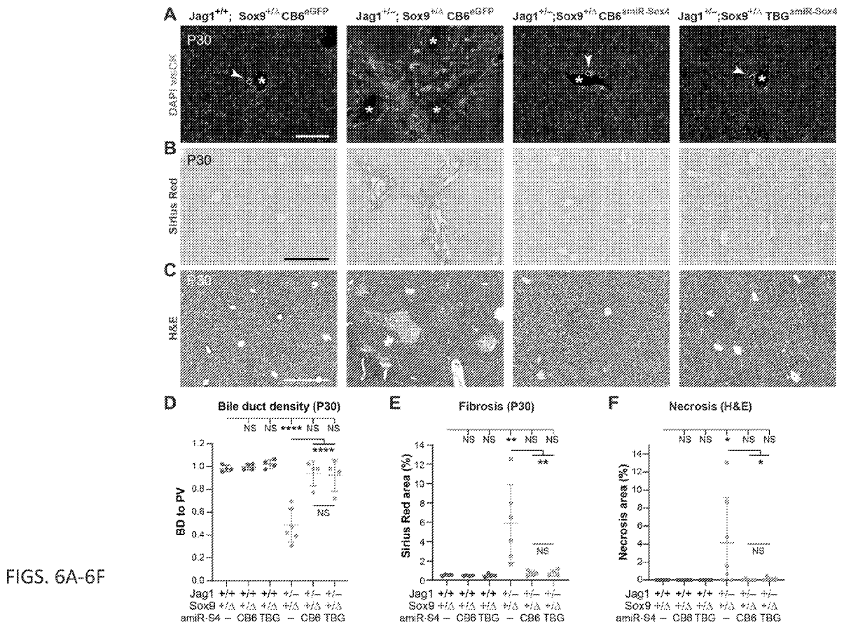

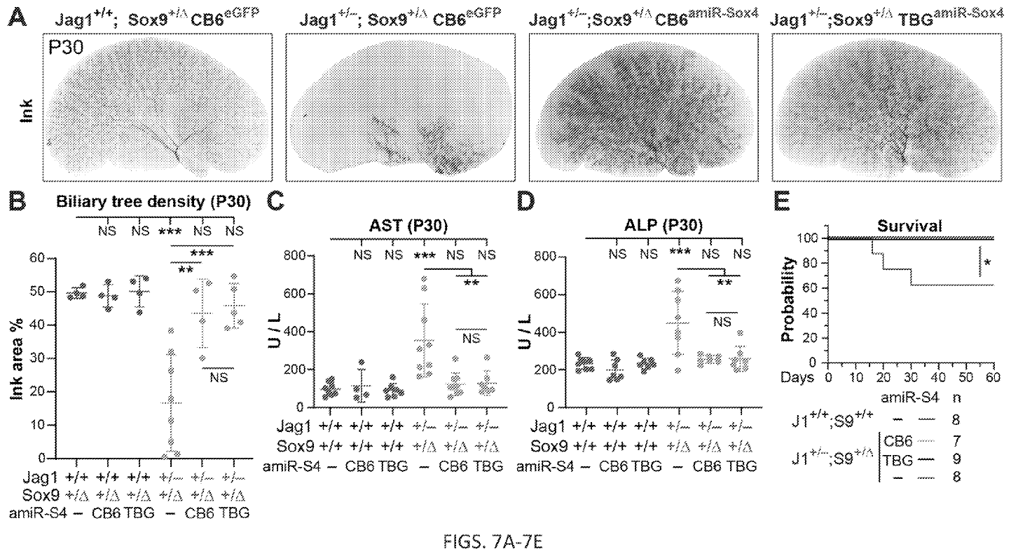

Next, it was examined if Sox4 knockdown can also improve the more severe liver phenotypes observed in Jag1^{+/-}; Sox9^{+/Δ} animals. Using the same injection protocol and dosage, AAV8-CB6-amiR-Sox4 and AAV8-TBG-amiR-Sox4 vectors were injected into Jag1^{+/-}; Sox9^{+/Δ} animals and harvested their livers at P30 for analysis. As shown in FIG. 6, BD formation and liver histology is improved dramatically upon injection of the AAV. Jag1^{+/-}; Sox9^{+/Δ} animals injected with control vector exhibit very large areas of ductular reaction and hypercellularity with significantly reduced biliary density, accompanied by extensive fibrosis and necrosis (FIG. 6). In contrast, the AAV-treated animals exhibit greatly reduced ductular reactions and many patent BDs, with their average BD to PV ratio increasing from an average of less than 0.5 to −0.9 for both AAVs, approaching the control ratio of −1.0 (FIG. 6A, 6D). Fibrosis was also improved upon injection of each AAV, as the Sirius Red⁺ liver area was decreased from an average of ˜6% in control Jag1^{+/-}; Sox9^{+/Δ} animals to average numbers less than 1%, which are statistically not different from the control levels (FIG. 6B, 6E). Similarly, AAV8-CB6-amiR-Sox4 and AAV8-TBG-amiR-Sox4 were both able to rescue the necrosis in Jag1^{+/-}; Sox9^{+/Δ} livers (FIG. 6C, 6F). Retrograde biliary ink injections of control and amiR-Sox4 AAV-injected animals show that Sox4 knockdown can dramatically improve the formation of the biliary tree. The biliary tree extends near the liver periphery in every AAV-treated animal examined (n=4 for AAV8-CB6-amiR-Sox4, n=5 for AAV8-TBG-amiR-Sox4), and the tree is dense and robust similar to a wild-type liver in most cases (FIG. 7A, 7B). This is in stark contrast to the rudimentary tree present in Jag1^{+/-}; Sox9^{+/Δ} animals injected with the control AAV8-CB6-eGFP vector (n=8) (FIG. 7A, 7B). This highlights the ability of the AAV to form a proper biliary tree, likely allowing these animals to drain bile so that it does not build up and cause damage. Serum chemistry analysis shows a significant increase in the level of aspartate aminotransferase (AST) and alkaline phosphatase (ALP) in Jag1^{+/-}; Sox9^{+/Δ} animals injected with the control vector (FIG. 7C, 7D), in agreement with a previous report o un-injected Jag1^{+/-}; Sox9^{+/Δ} animals (19). Again, the amiR-Sox4 AAVs both rescued this phenotype (FIG. 7A, 7B). Some lethality was noticed in Jag1^{+/-}; Sox9^{+/Δ} animals injected with the control vector, which was fully rescued by both amiR-Sox4 AAVs (FIG. 7E).

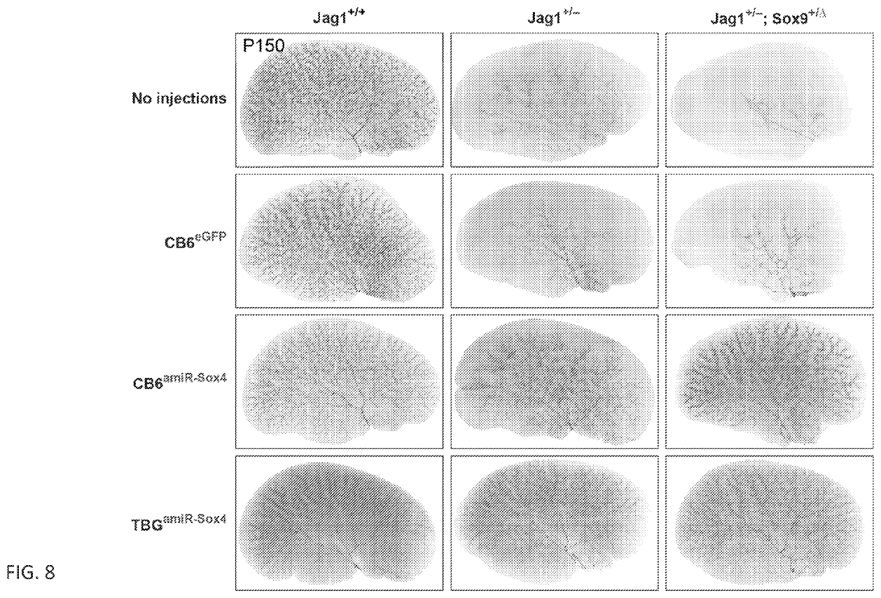

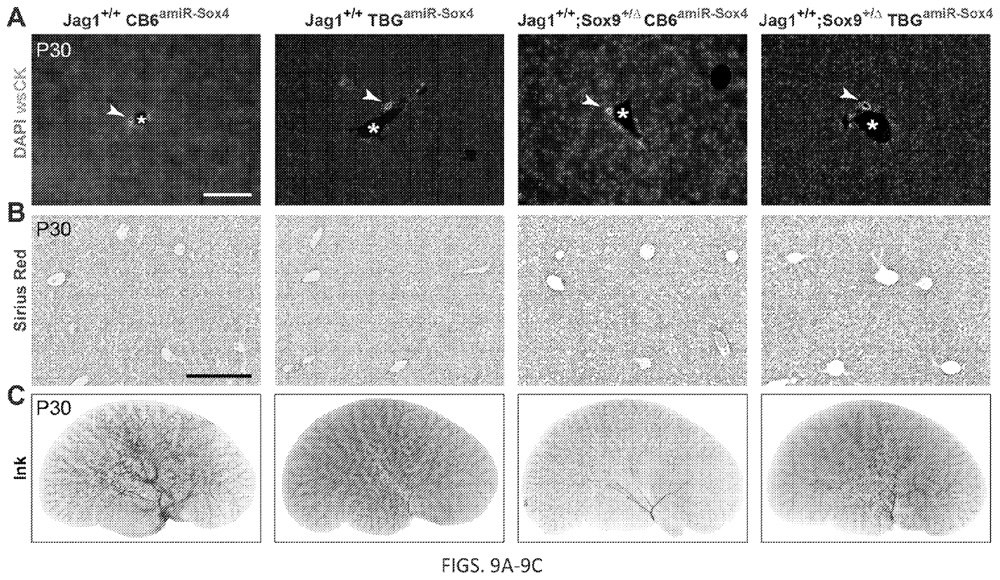

To determine the long-term impact of the single AAV injections at P1 on biliary tree structure of ALGS models and control mice, additional cohorts were generated of WT (Jag1^{+/-}), Jag1^{+/-}, and Jag1^{+/-}; Sox9^{+/Δ} animals with no injections, or injected with AAV8-CB6-eGFP, AAV8-CB6-amiR-Sox4, or AAV8-TBG-amiR-Sox4, and they were aged well into adulthood. Ink injections into the common bile duct of these animals showed that even 5-6 months after injection, the biliary tree in Sox4 knockdown animals still covers a lot more of the liver compared to un-injected animals and those injected with the control AAV (FIG. 8). Finally, injection of AAV8-CB6-amiR-Sox4 and AAV8-TBG-amiR-Sox4 vectors into control animals did not lead to any adverse effects on biliary development and liver function (FIGS. 5-9). It was concluded that one IP injection of AAV8-amiR-Sox4 vectors driving Sox4 knockdown with a ubiquitous promoter or only in TBG⁺ cells is sufficient to dramatically improve the severe liver phenotypes of the Jag1^{+/-}; Sox9^{+/Δ}, model.

All of the AAV rescue experiments shown so far were generated by IP injection of AAV vectors into mice and by using a specific small silencing RNA against Sox4. To examine whether the observed rescue can be achieved by targeting a different region of Sox4 and also a different injection route, an AAV8-CB6-amiR-Sox4 vector was generated containing the artificial microRNA harboring a second, non-overlapping shRNA against Sox4 (AAV8-CB6-amiR-Sox4. #2). A single IP injection of 2×10¹¹ genomes of this vector showed a strong rescue of Jag1^{+/-} liver phenotypes as well (FIG. 10A and not shown). Similarly, retro-orbital (intravenous) injection of 2×10¹¹ genomes of the first AAV8-CB6-amiR-Sox4 vector from FIG. 4A also led to significant improvement of liver phenotypes in Jag1^{+/-} mice (FIG. 10B and not shown). These observations establish that the rescue of ALGS phenotypes in the model is not limited to a specific targeting sequencing or mode of delivery.

Altogether, the preclinical data indicate that even after BD paucity has already affected ALGS mouse models with moderate to severe liver involvement, reducing Sox4 levels in the liver can reverse the course of the ALGS liver disease. Moreover, the work indicates that Sox4 knockdown in TBG⁺ cells in the liver leads to rescue levels comparable to or even better than those achieved by ubiquitous Sox4 knockdown. Accordingly, in specific embodiments AAV8-mediated knockdown of human SOX4 is a promising therapeutic for ALGS liver disease at least for two reasons: (1) It is likely to help the affected children form a robust biliary tree in their own native liver, thereby avoiding the damage that leads to liver failure and steering the patients away from the path of liver transplantation; and (2) The therapeutic SOX4 knockdown can be limited to a specific cell type in the liver, thereby not affecting SOX4 expression in most cells of the body and dramatically reducing the chance for side effects.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. D. Alagille, M. Odievre, M. Gautier, J. P. Dommergues, Hepatic ductular hypoplasia associated with characteristic facies, vertebral malformations, retarded physical, mental, and sexual development, and cardiac murmur. J Pediair 86, 63-71 (1975).
2. K. M. Emerick, E. B. Rand, E. Goldmuntz, I. D. Krantz, N. B. Spinner, D. A. Piccoli, Features of Alagille syndrome in 92 patients: frequency and relation to prognosis. Hepatology 29, 822-829 (1999).
3. L. Li, I. D. Krantz, Y. Deng, A. Genin, A. B. Banta, C. C. Collins, M. Qi, B. J. Trask, W. L. Kuo, J. Cochran, T. Costa, M. E. Pierpont, E. B. Rand, D. A. Piccoli, L. Hood, N. B. Spinner, Alagille syndrome is caused by mutations in human Jagged1, which encodes a ligand for Notch1. Nature genetics 16, 243-251 (1997).
4. T. Oda, A. G. Elkahloun, B. L. Pike, K. Okajima, I. D. Krantz, A. Genin, D. A. Piccoli, P. S. Meltzer, N. B. Spinner, F. S. Collins, S. C. Chandrasekharappa, Mutations in the human Jagged1 gene are responsible for Alagille syndrome. Nature genetics 16, 235-242 (1997).
5. D. M. Warthen, E. C. Moore, B. M. Kamath, J. J. Morrissette, P. A. Sanchez-Lara, D. A. Piccoli, I. D. Krantz, N. B. Spinner, Jagged1 (JAG1) mutations in Alagille syndrome: increasing the mutation detection rate. Human mutation 27, 436-443 (2006).
6. M. A. Gilbert, R. C. Bauer, R. Rajagopalan, C. M. Grochowski, G. Chao, D. McEldrew, J. A. Nassur, E. B. Rand, B. L. Krock, B. M. Kamath, I. D. Krantz, D. A. Piccoli, K. M. Loomes, N. B. Spinner, Alagille syndrome mutation update: Comprehensive overview of JAG1 and NOTCH2 mutation frequencies and insight into missense variant classification. Human mutation 40, 2197-2220 (2019).
7. B. M. Kamath, R. C. Bauer, K. M. Loomes, G. Chao, J. Gerfen, A. Hutchinson, W. Hardikar, G. Hirschfield, P. Jara, 1. D. Krantz, P. Lapunzina, L. Leonard, S. Ling, V. L. Ng, P. L. Hoang, D. A. Piccoli, N. B. Spinner, NOTCH2 mutations in Alagille syndrome. Journal of medical genetics 49, 138-144 (2012).
8. R. McDaniell, D. M. Warthen, P. A. Sanchez-Lara, A. Pai, I. D. Krantz, D. A. Piccoli, N. B. Spinner, NOTCH2 mutations cause Alagille syndrome, a heterogeneous disorder of the notch signaling pathway. American journal of human genetics 79, 169-173 (2006).
9. M. Mouzaki, L. M. Bass, R. J. Sokol, D. A. Piccoli, C. Quammie, K. M. Loomes, J. E. Heubi, P. M. Hertel, R. Scheenstra, K. Furuya, E. Kutsch, N. B. Spinner, K. N. Robbins, V. Venkat, P. Rosenthal, J. Beyene, A. Baker, B. M. Kamath, Early life predictive markers of liver disease outcome in an International, Multicentre Cohort of children with Alagille syndrome. Liver international: official journal of the International Association for the Study of the Liver 36, 755-760 (2016).
10. E. Gonzales, W. Hardikar, M. Stormon, A. Baker, L. Hierro, D. Gliwicz, F. Lacaille, A. Lachaux, E. Sturm, K. D. R. Setchell, C. Kennedy, A. Dorenbaum, J. Steinmetz, N. K. Desai, A. J. Wardle, W. Garner, P. Vig, T. Jaecklin, E. M. Sokal, E. Jacquemin, Efficacy and safety of maralixibat treatment in patients with Alagille syndrome and cholestatic pruritus (ICONIC): a randomised phase 2 study. Lancet 398, 1581-1592 (2021).
11. M. D. Ayoub, B. M. Kamath, Alagille Syndrome: Diagnostic Challenges and Advances in Management. Diagnostics (Basel) 10, 907 (2020).
12. B. M. Kamath, K. B. Schwarz, N. Hadzic, Alagille syndrome and liver transplantation. J Pediatr Gastroenterol Nutr 50, 11-15 (2010).
13. B. M. Kamath, W. Yin, H. Miller, R. Anand, E. B. Rand, E. Alonso, J. Bucuvalas, T. Studies of Pediatric Liver, Outcomes of liver transplantation for patients with Alagille syndrome: the studies of pediatric liver transplantation experience. Liver transplantation official publication of the American Association for the Study of Liver Diseases and the International Liver Transplantation Society 18, 940-948 (2012).
14. B. M. Kamath, A. Baker, R. Houwen, L. Todorova, N. Kerkar, Systematic Review: The Epidemiology, Natural History, and Burden of Alagille Syndrome. J Pediatr Gastroenterol Nutr 67, 148-156 (2018).
15. B. M. Kamath, W. Ye, N. P. Goodrich, K. M. Loomes, R. Romero, J. E. Heubi, D. H. Leung, N. B. Spinner, D. A. Piccoli, E. M. Alonso, S. L. Guthery, S. J. Karpen, C. L. Mack, J. P. Molleston, K. F. Murray, P. Rosenthal, J. E. Squires, J. Teckman, K. S. Wang, R. Thompson, J. C. Magee, R. J. Sokol, N. Childhood Liver Disease Research, Outcomes of Childhood Cholestasis in Alagille Syndrome: Results of a Multicenter Observational Study. Hepatol Commun 4, 387-398 (2020).
16. S. M. Vandriel, L. T. Li, H. She, J. S. Wang, M. A. Gilbert, I. Jankowska, P. Czubkowski, D. Gliwicz-Miedzinska, E. M. Gonzales, E. Jacquemin, J. Bouligand, N. B. Spinner, K. M. Loomes, D. A. Piccoli, L. D′Antiga, E. Nicastro, E. Sokal, T. Demaret, N. H. Ebel, J. A. Feinstein, R. Fawaz, S. Nastasio, F. Lacaille, D. Debray, H. Arnell, B. Fischler, S. Siew, M. Stormon, S. J. Karpen, R. Romero, K. M. Kim, W. Y. Baek, W. Hardikar, S. Shankar, A. J. Roberts, H. M. Evans, M. K. Jensen, M. Kavan, S. S. Sundaram, A. Chaidez, P. Karthikeyan, M. C. Sanchez, M. L. Cavalieri, H. J. Verkade, W. S. Lee, J. E. Squires, C. Hajinicolaou, C. Lertudomphonwanit, R. T. Fischer, C. Larson-Nath, Y. Mozer-Glassberg, C. Arikan, H. C. Lin, J. Q. Bernabeu, S. Alam, D. A. Kelly, E. Carvalho, C. T. Ferreira, G. Indolfi, R. E. Quiros-Tejeira, P. Bulut, P. L. Calvo, Z. Onal, P. L. Valentino, D. M. Desai, J. Eshun, M. Rogalidou, A. Dezsofi, S. Wiecek, G. Nebbia, R. B. Pinto, V. M. Wolters, M. L. Tamara, A. N. Zizzo, J. Garcia, K. Schwarz, M. Beretta, T. D. Sandahl, C. Jimenez-Rivera, N. Kerkar, J. Brecelj, Q. Mujawar, N. Rock, C. M. Busoms, W. Karnsakul, E. Lurz, E. Santos-Silva, N. Blondet, L. Bujanda, U. Shah, R. J. Thompson, B. E. Hansen, B. M. Kamath, A. A. S. G. Global, Natural history of liver disease in a large international cohort of children with Alagille syndrome: Results from the GALA study. Hepatology 77, 512-529 (2023).
17. Y. Xue, X. Gao, C. E. Lindsell, C. R. Norton, B. Chang, C. Hicks, M. Gendron-Maguire, E. B. Rand, G. Weinmaster, T. Gridley, Embryonic lethality and vascular defects in mice lacking the Notch ligand Jagged1. Human molecular genetics 8, 723-730 (1999).
18. S. M. Thakurdas, M. F. Lopez, S. Kakuda, R. Fernandez-Valdivia, N. Zarrin-Khameh, R. S. Haltiwanger, H. Jafar-Nejad, Jagged1 heterozygosity in mice results in a congenital cholangiopathy which is reversed by concomitant deletion of one copy of Poglutl (Rumi). Hepatology 63, 550-565 (2016).
19. J. M. Adams, K. A. Huppert, E. C. Castro, M. F. Lopez, N. Niknejad, S. Subramanian, N. Zarrin-Khameh, M. J. Finegold, S. S. Huppert, H. Jafar-Nejad, Sox9 is a modifier of the liver disease severity in a mouse model of Alagille syndrome. Hepatology 71, 1331-1349 (2020).
20. C. Postic, M. Shiota, K. D. Niswender, T. L. Jetton, Y. Chen, J. M. Moates, K. D. Shelton, J. Lindner, A. D. Cherrington, M. A. Magnuson, Dual roles for glucokinase in glucose homeostasis as determined by liver and pancreatic beta cell-specific gene knock-outs using Cre recombinase. J Biol Chem 274, 305-315 (1999).
21. F. Geisler, F. Nagl, P. K. Mazur, M. Lee, U. Zimber-Strobl, L. J. Strobl, F. Radtke, R. M. Schmid, J. T. Siveke, Liver-specific inactivation of Notch2, but not Notch1, compromises intrahepatic bile duct development in mice. Hepatology 48, 607-616 (2008).
22. A. Poncy, A. Antoniou, S. Cordi, C. E. Pierreux, P. Jacquemin, F. P. Lemaigre, Transcription factors SOX4 and SOX9 cooperatively control development of bile ducts. Dev Biol 404, 136-148 (2015).
23. A. Penzo-Mendez, P. Dy, B. Pallavi, V. Lefebvre, Generation of mice harboring a Sox4 conditional null allele. Genesis 45, 776-780 (2007).
24. Z. D. Goodman, Grading and staging systems for inflammation and fibrosis in chronic liver diseases. J Hepatol 47, 598-607 (2007).
25. J. R. Schaub, K. A. Huppert, S. N. T. Kurial, B. Y. Hsu, A. E. Cast, B. Donnelly, R. A. Karns, F. Chen, M. Rezvani, H. Y. Luu, A. N. Mattis, A. L. Rougemont, P. Rosenthal, S. S. Huppert, H. Willenbring, De novo formation of the biliary system by TGFbeta-mediated hepatocyte transdifferentiation. Nature 557, 247-251 (2018).
26. J. Xie, P. W. L. Tai, A. Brown, S. Gong, S. Zhu, Y. Wang, C. Li, C. Colpan, Q. Su, R. He, H. Ma, J. Li, H. Ye, J. Ko, P. D. Zamore, G. Gao, Effective and Accurate Gene Silencing by a Recombinant AAV-Compatible MicroRNA Scaffold. Mol Ther 28, 422-430 (2020).
27. L. Bartalena, J. R. Tata, J. Robbins, Characterization of nascent and secreted thyroxine-binding globulin in cultured human hepatoma (Hep G2) cells. J Biol Chem 259, 13605-13609 (1984).
28. S. Shin, K. J. Wangensteen, M. Teta-Bissett, Y. J. Wang, E. Mosleh-Shirazi, E. L. Buza, L. E. Greenbaum, K. H. Kaestner, Genetic lineage tracing analysis of the cell of origin of hepatotoxin-induced liver tumors in mice. Hepatology 64, 1163-1177 (2016).
29. L. Wang, H. Wang, P. Bell, D. McMenamin, J. M. Wilson, Hepatic gene transfer in neonatal mice by adeno-associated virus serotype 8 vector. Hum Gene Ther 23, 533-539 (2012).
30. K. Yanger, Y. Zong, L. R. Maggs, S. N. Shapira, R. Maddipati, N. M. Aiello, S. N. Thung, R. G. Wells, L. E. Greenbaum, B. Z. Stanger, Robust cellular reprogramming occurs spontaneously during liver regeneration. Genes Dev 27, 719-724 (2013).

What is claimed is:

1. A method of treating bile duct paucity in an individual, comprising administering to the individual an effective amount of one or more agents that reduces expression of SRY-Box Transcription Factor 4 (SOX4) gene and/or activity of SOX4 protein.

2. The method of claim 1, wherein the individual has neonatal cholestasis.

3. The method of claim 1 or 2, wherein the individual has Alagille syndrome (ALGS).

4. The method of any one of claims 1-3, wherein the agent partially or fully reduces expression of SOX4.

5. The method of any one of claims 1-4, wherein the agent is a nucleic acid, protein, small molecule, or combination thereof.

6. The method of any one of claims 1-5, wherein the agent is a nucleic acid that targets expression of SOX4.

7. The method of any one of claims 1-6, wherein the agent is an shRNA that targets expression of SOX4.

8. The method of any one of claims 1-5, wherein the agent is a SOX4 antibody or functional fragment thereof.

9. The method of claim 8 wherein the antibody is polyclonal or monoclonal.

10. The method of any one of claims 1-9 wherein the individual is in utero or is a neonate, infant, child, or adult.

11. The method of any one of claims 1-10, wherein the individual has cholestasis, fibrosis, necrosis, and/or ductular reactions.

12. The method of any one of claims 1-11 wherein the agent is a nucleic acid in a vector.

13. The method of claim 12, wherein the vector is a viral vector.

14. The method of claim 12, wherein the vector is a non-viral vector.

15. The method of claim 13, wherein the viral vector is an adeno-associated viral vector, an adenoviral vector, a lentiviral vector, or a retroviral vector.

16. The method of claim 15, wherein the adeno-associated viral vector is an adeno-associated virus 8 (AAV8) vector.

17. The method of any one of claims 1-16, wherein the individual is in need of a liver transplant or is at risk for needing a liver transplant.

18. The method of any one of claims 1-17, wherein the individual has liver failure.

19. The method of any one of claims 1-18, wherein the individual has a mutation in a gene associated with ALGS.

20. The method of any one of claims 1-19, wherein the individual has an autosomal dominant mutation in the JAG1 gene.

21. The method of any one of claims 1-20, wherein the individual has an autosomal dominant mutation in the NOTCH2 gene.

22. The method of any one of claims 1-21, wherein the individual has had surgery for the bile duct paucity or will have surgery for the bile duct paucity.

23. The method of any one of claims 1-22, wherein enterohepatic bile acid transport in the individual has been or will be modified through one or more inhibitors and/or one or more binding resins.

24. The method of any one of claims 1-23, wherein the individual has ALGS that has affected the cardiovascular system, kidneys, eye, and/or skeleton of the individual.


SEQUENCE LISTING (4 sequences)

SEQ ID NO: 1 - DNA, 4869 bp, Homo sapiens
  source 1..4869: mol_type = genomic DNA
gctctaagctgcagcaagagaaactgtgtgtgaggggaagaggcctgtttcgctgtcgggtctctagttcttgcacgctctttaagagtctgcactggaggaactcctgccattaccagctcccttcttgcagaagggagggggaaacatacatttattcatgccagtctgttgcatgcaggctttttggcttcctaccttgcaacaaaataattgcaccaactccttagtgccgattccgcccacagagagtcctggagccacagtcttttttgctttgcattgtaggagagggactaagtgctagagactatgtcgctttcctgagctaccgagagcgctcgtgaactggaatcaactgcttcagggaaaaagaaaaaaaaaaaaaaaagacttgcctgggaggccgcgagaaacttgcattggaagcttcagcaaccagcattcgagaaactcctctctactttagcacggtctccagactcagccgagagacagcaaactgcagcgcggtgagagagcgagagagagggagagagagactctccagcctgggaactataactcctctgcgagaggcggagaactccttccccaaatcttttggggacttttctctctttacccacctccgcccctgcgaggagttgaggggccagttcggccgccgcgcgcgtcttcccgttcggcgtgtgcttggcccggggaaccgggagggcccggcgatcgcgcggcggccgccgcgagggtgtgagcgcgcgtgggcgcccgccgagccgaggccatggtgcagcaaaccaacaatgccgagaacacggaagcgctgctggccggcgagagctcggactcgggcgccggcctcgagctgggaatcgcctcctcccccacgcccggctccaccgcctccacgggcggcaaggccgacgacccgagctggtgcaagaccccgagtgggcacatcaagcgacccatgaacgccttcatggtgtggtcgcagatcgagcggcgcaagatcatggagcagtcgcccgacatgcacaacgccgagatctccaagcggctgggcaaacgctggaagctgctcaaagacagcgacaagatccctttcattcgagaggcggagcggctgcgcctcaagcacatggctgactaccccgactacaagtaccggcccaggaagaaggtgaagtccggcaacgccaactccagctcctcggccgccgcctcctccaagccgggggagaagggagacaaggtcggtggcagtggcgggggcggccatgggggcggcggcggcggcgggagcagcaacgcggggggaggaggcggcggtgcgagtggcggcggcgccaactccaaaccggcgcagaaaaagagctgcggctccaaagtggcgggcggcgcgggcggtggggttagcaaaccgcacgccaagctcatcctggcaggcggcggcggcggcgggaaagcagcggctgccgccgccgcctccttcgccgccgaacaggcgggggccgccgccctgctgcccctgggcgccgccgccgaccaccactcgctgtacaaggcgcggactcccagcgcctcggcctccgcctcctcggcagcctcggcctccgcagcgctcgcggccccgggcaagcacctggcggagaagaaggtgaagcgcgtctacctgttcggcggcctgggcacgtcgtcgtcgcccgtgggcggcgtgggcgcgggagccgaccccagcgaccccctgggcctgtacgaggaggagggcgcgggctgctcgcccgacgcgcccagcctgagcggccgcagcagcgccgcctcgtcccccgccgccggccgctcgcccgccgaccaccgcggctacgccagcctgcgcgccgcctcgcccgccccgtccagcgcgccctcgcacgcgtcctcctcggcctcgtcccactcctcctcttcctcctcctcgggctcctcgtcctccgacgacgagttcgaagacgacctgctcgacctgaaccccagctcaaactttgagagcatgtccctgggcagcttcagttcgtcgtcggcgctcgaccgggacctggattttaacttcgagcccggctccggctcgcacttcgagttcccggactactgcacgcccgaggtgagcgagatgatctcgggagactggctcgagtccagcatctccaacctggttttcacctactgaagggcgcgcaggcagggagaagggccggggggggtaggagaggagaaaaaaaaagtgaaaaaaagaaacgaaaaggacagacgaagagtttaaagagaaaagggaaaaaagaaagaaaaagtaagcagggctggcttcgcccgcgttctcgtcgtcggatcaaggagcgcggcggcgttttggacccgcgctcccatcccccaccttcccgggccggggacccactctgcccagccggagggacgcggaggaggaagagggtagacaggggcgacctgtgattgttgttattgatgttgttgttgatggcaaaaaaaaaaaagcgacttcgagtttgctcccctttgcttgaagagaccccctcccccttccaacgagcttccggacttgtctgcacccccagcaagaaggcgagttagttttctagagacttgaaggagtctcccccttcctgcatcaccaccttggttttgttttattttgcttcttggtcaagaaaggaggggagaacccagcgcacccctccccccctttttttaaacgcgtgatgaagacagaaggctccggggtgacgaatttggccgatggcagatgttttgggggaacgccgggactgagagactccacgcaggcgaattcccgtttggggcttttttttcctccctcttttccccttgccccctctgcagccggaggaggagatgttgaggggaggaggccagccagtgtgaccggcgctaggaaatgacccgagaaccccgttggaagcgcagcagcgggagctaggggcgggggcggaggaggacacgaactggaagggggttcacggtcaaactgaaatggatttgcacgttggggagctggcggcggcggctgctgggcctccgccttcttttctacgtgaaatcagtgaggtgagacttcccagaccccggaggcgtggaggagaggagactgtttgatgtggtacaggggcagtcagtggagggcgagtggtttcggaaaaaaaaaaagaaaaaaagaaaaaaaaagaaaaaaaaaagatttttttcttctcttaatcggaatcgtgatggtgttggattatttcaatggtggggttaatatagcatgttatcctgtctatcttttaaagatttctgtataagactgttgagcagtttttaaaatagtgtaggataatataaaaagcagatagatggcgctatgtttgattcctacaacgaaattatcaccagctttttttcattcttaactctttaaaggattcaaacgcaactcaaatctgtgctggactttaaaaaaacaattcaggaccaaattttttctcagtgtgtgtgtttattccttataggtgtaaatgagaagacgtgtttttttccttcaccgatgctccatcctcgtatttctttttccttgtaaatgtaatcagatgccattttatatgtggacgtatttatactggccaaacatattttttcttttgtccctttttttctttcctttctttttacttcctttatttctttattccttccttttcctttttttcttttttttttctttttttttttttttttttggtagttgttgttacccacgccattttacgtctccttcactgaagggctagagttttaacttttaattttttatatttaaatgtagacttttgacacttttaaaaaacaaaaaaagacaagagagatgaaaacgtttgattattttctcagtgtatttttgtaaaaaatatataaagggggtgttaatcggtgtaaatcgctgtttggatttcctgattttataacagggcggctggttaatatctcacacagtttaaaaaatcagcccctaatttctccatgtttacacttcaatctgcaggcttcttaaagtgacagtatcccttaacctgccaccagtgtccaccctccggcccccgtcttgtaaaaaggggaggagaattagccaaacactgtaagcttttaagaaaaacaaagttttaaacgaaatactgctctgtccagaggctttaaaactggtgcaattacagcaaaaagggattctgtagctttaacttgtaaaccacatcttttttgcactttttttataagcaaaaacgtgccgtttaaaccactggatctatctaaatgccgatttgagttcgcgacactatgtactgcgtttttcattcttgtatttgactatttaatcctttctacttgtcgctaaatataattgttttagtcttatggcatgatgatagcatatgtgttcaggtttatagctgttgtgtttaaaaattgaaaaaagtggaaaacatctttgtacatttaagtctgtattataataagcaaaaagattgtgtgtatgtatgtttaatataacatgacaggcactaggacgtctgcctttttaaggcagttccgttaagggtttttgtttttaaacttttttttgccatccatcctgtgcaatatgccgtgtagaatatttgtcttaaaattcaaggccacaaaaacaatgtttgggggaaaaaaaagaaaaaatcatgccagctaatcatgtcaagttcactgcctgtcagattgttgatatataccttctgtaaataactttttttgagaaggaaataaaatcagctggaactgaaccctaaa

SEQ ID NO: 2 - protein, 474 aa, Homo sapiens
MVQQTNNAENTEALLAGESSDSGAGLELGIASSPTPGSTASTGGKADDPSWCKTPSGHIKRPMNAFMVWSQIERRKIMEQSPDMHNAEISKRLGKRWKLLKDSDKIPFIREAERLRLKHMADYPDYKYRPRKKVKSGNANSSSSAAASSKPGEKGDKVGGSGGGGHGGGGGGGSSNAGGGGGGASGGGANSKPAQKKSCGSKVAGGAGGGVSKPHAKLILAGGGGGGKAAAAAAASFAAEQAGAAALLPLGAAADHHSLYKARTPSASASASSAASASAALAAPGKHLAEKKVKRVYLFGGLGTSSSPVGGVGAGADPSDPLGLYEEEGAGCSPDAPSLSGRSSAASSPAAGRSPADHRGYASLRAASPAPSSAPSHASSSASSHSSSSSSSGSSSSDDEFEDDLLDLNPSSNFESMSLGSFSSSSALDRDLDFNFEPGSGSHFEFPDYCTPEVSEMISGDWLESSISNLVFTY

SEQ ID NO: 3 - DNA, 269 bp, synthetic construct
  source 1..269: mol_type = other DNA
agggctctgcgtttgctccaggtagtccgctgctcccttgggcctgggcccactgacagccctggtgcctctggccggctgcacacctcctggcgggcagctgtgtcgtcttcgaactcgtcgtcgtgttctggcaatacctgcgacgacgtctccgaagacgacacggaggcctgccctgactgcccacggtgccgtggccaaagaggatctaagggcaccgctgagggcctacctaaccatcgtggggaataaggacagtgtcaccc

SEQ ID NO: 4 - DNA, 269 bp, synthetic construct
  source 1..269: mol_type = other DNA
agggctctgcgtttgctccaggtagtccgctgctcccttgggcctgggcccactgacagccctggtgcctctggccggctgcacacctcctggcgggcagctgtgtaaacctgaacacatatgctatgttctggcaatacctgtagcatatcagctcaggtttacacggaggcctgccctgactgcccacggtgccgtggccaaagaggatctaagggcaccgctgagggcctacctaaccatcgtggggaataaggacagtgtcaccc